(19) United States
(12) Patent Application Publication
(10) Pub. No.: US 2025/0025432 A1
(43) Pub. Date: Jan. 23, 2025

(54) METHODS FOR IMPROVING DRIVING BEHAVIOR

(71) Applicant: Tris Pharma, Inc., Monmouth Junction, NJ (US)

(72) Inventors: Antonio Pardo, Monmouth Junction, NJ (US); Jim Potenziano, Monmouth Junction, NJ (US)

(21) Appl. No.: 18/589,177

(22) Filed: Feb. 27, 2024

Related U.S. Application Data
(60) Provisional application No. 63/487,158, filed on Feb. 27, 2023.

(51) Int. Cl. A61K 31/137; A61K 9/00; A61K 9/28; A61P 43/00

(52) U.S. Cl. CPC A61K 31/137; A61K 9/0002; A61K 9/28; A61P 43/00

(57) ABSTRACT

Use of an oral amphetamine extended release solid dose for improving driving skills in adults is described. The compositions contain a combination of an uncoated amphetamine-cation exchange resin complex, a barrier coated amphetamine-cation exchange resin complex-matrix, and an uncomplexed amphetamine, wherein one or more of these components contains blends of different forms of amphetamines. Either the modified release coated and/or the uncoated amphetamine-cation exchange resin complex may have two forms of amphetamine in a complex with a single cation exchange resin. Following administration of a single dose of the composition, a therapeutically effective amount of amphetamine is reached by about one hour and the composition provides at least a thirteen hour effect post-dose.

### BACKGROUND OF THE INVENTION

The literature documents that Attention Deficit Hyperactivity Disorder (ADHD) is a significant risk for motor vehicle accidents and impaired driving. In several studies, individuals with ADHD self-reported significantly more traffic citations, particularly for speeding, vehicular crashes and license suspensions than controls. Young adults with ADHD have also been shown to be at increased risk for motor vehicle accidents and motor vehicle-related injuries and fatalities, traffic citations for speeding, and license suspensions in several other studies. Barkley R A, Cox D J. A review of driving risks and impairments associated with attention-deficit/hyperactivity disorder and the effects of stimulant medication on driving performance. J Safety Res. 2007; 38(1):113-128.

Weiss et al. (2007) reported that adolescents and young adults with ADHD were more likely to experience traffic accidents. Amanda L. Thompson, et al, Risky Driving in Adolescents and Young Adults with Childhood ADHD (2007), Journal of Pediatric Psychology vol. 32 no. 7, 745-759. Barkley et al. (2007) reported that young adults with ADHD were more likely to be involved in traffic accidents, to be at fault in automobile accidents and to have received more traffic citations than normal controls. A New Zealand clinical trial of children with ADHD found similar results showing that individuals with ADHD had significantly more driving transgressions than controls [Nada-Raja, S., et al, (1997). Inattentive and hyperactive behaviors and driving offenses in adolescence. Journal of the American Academy of Child and Adolescent Psychiatry, 36(4), 515-522] and young adults with ADHD were at a greater risk to have traffic accidents involving injuries and traffic violations. Woodward L J1, et al, Driving outcomes of young people with attentional difficulties in adolescence. J Am Acad Child Adolesc Psychiatry. 2000 May; 39(5):627-34.

The reasons for ADHD-associated impaired driving are not entirely clear. Treat et al. (1977) purported that cognitive abilities play a significant role in driving risks, and found that the cognitive factors of inattention and distractibility, which also characterize ADHD diagnoses, are to blame. Treat, J. R., et al, Tri-Level Study of the Causes of Traffic Accidents Vol. I: Causal Factor Tabulations and Assessment. Washington D. C.: U. S. Department of Transportation. Alternatively, excessive speed has been documented in numerous studies of self-report [Barkley 2007] as one of the risky driving behaviors in individuals with ADHD, though driving simulator studies of ADHD driving have neglected to look at driving speed. [Barkley 2007]. In line with these findings, it is plausible to hypothesize that adults with ADHD often speed because they are easily distracted by competing stimuli while driving (e.g., radio, conversation, cell phone), and are therefore unable to regulate their speed. Laberge, J., et al., (2005). Driving skills among ADHD drivers: Preliminary research. Paper presented at the Third International Conference on Traffic and Transport Psychology.

Considering that the presence of ADHD is a risk factor for car accidents, and car accidents are a leading cause of mortality in the young, there is a need for improving understanding of the causes of driving impairments and diminishing the risk for serious car accidents and driving associated deaths.

### SUMMARY OF THE INVENTION

Methods are provided herein for improving driving behavior and reducing traffic accidents in a human subject operating a motor vehicle. In certain embodiments, the subjects have attention deficit hyperactivity disorder (ADHD) or a related condition, and the method involves dosing the subject once daily with an amphetamine extended release tablet provided herein. In certain embodiments, a method for decreasing the risk of a driving accident in a human operating a motor vehicle is provided. In certain embodiments, the method involves providing the subject with an amphetamine extended release tablet composition as provided herein. In certain embodiments, improvements in driving behavior are obtained within about 45 minutes post-dosing and through about 10 hours post-dosing. In certain embodiments, improvements may be observed in less than 45 minutes and for longer than 10 hours post-dosing.

Amphetamine extended release compositions are described which provides in a single composition an immediate release of amphetamine with a fast onset of therapeutic effect and a therapeutic effect through an extended release. In certain embodiments, the therapeutic effect lasts up to about 13 hours. In certain embodiments, the compositions useful in the methods provided herein provide the extended release therapeutic effect with a formulation providing a higher percentage of immediate release components as compared to extended release components. Further, in certain embodiments, the compositions have a single plasma concentration peak for both d-amphetamine and l-amphetamine post-dosing. Tablets which are both chewable and which may also be orally disintegrating, orally dissolvable, and/or dispersible, are provided. These compositions provide easy administration for patients who have difficulty swallowing pills, especially for the pediatric and geriatric patients. Further because chewing does not alter the extended release properties of the product, patients have option to swallow, dissolve in the mouth (buccal cavity) or to chew a solid unit dose tablet without affecting the release property of the product. Additionally, the compositions can be dose titrated without altering the release profile of the composition, i.e., by splitting the tablet. Methods of treating patients in need thereof with these amphetamine (AMP) extended release compositions are further provided.

In one aspect, the methods utilize an orally administrable extended release amphetamine tablet comprising: an immediate release and an extended therapeutic effect for d-amphetamine and for l-amphetamine, a single plasma concentration peak for d-amphetamine and for l-amphetamine, and wherein the immediate release component comprises greater than 60% w/w immediate release amphetamines based on the total weight of amphetamines in the tablet, and wherein the tablet further comprises: (A) a modified release amphetamine component which comprises at least one modified release barrier coated amphetamine-cation exchange resin complex-optional matrix which comprises (i) two or more amphetamines bound to the same cation exchange resin or each bound to a different cation exchange resin, wherein when the optional matrix is present, the amphetamine-cation exchange resin complex-matrix further comprises a hydrophilic polymer or copolymer or a hydrophobic polymer and (ii) a water-insoluble, water-permeable, non-ionic modified release barrier coating which provides a modified release to the two or more amphetamines, wherein the two or more amphetamines are at least a d-amphetamine and an l-amphetamine, wherein the d-amphetamine and the l-amphetamine are provided by d-amphetamine and at least one of (d,l)-amphetamine and l-amphetamine; and (B) a first immediate release amphetamine component which comprises d-amphetamine or a pharmaceutically acceptable salt thereof, and l-amphetamine or a pharmaceutically acceptable salt thereof, or mixtures thereof, wherein the d- and l-amphetamine are provided by d-amphetamine and at least one of (d,l)-amphetamine and l-amphetamine; (C) a second immediate release amphetamine component which comprises an amphetamine-cation exchange resin complex in an optional matrix, wherein the amphetamine-cation exchange resin complex-optional matrix comprises at least a d-amphetamine and an l-amphetamine both bound to the same cation exchange resin or each bound to different cation exchange resins, wherein the d-amphetamine and the l-amphetamine are provided by d-amphetamine and at least one of (d,l)-amphetamine or l-amphetamine. The composition contains two counterions to the amphetamines. The immediate release, modified release, titratable amphetamine composition is a chewable tablet, which is optionally scored for accurate, easy division or splitting. In such an embodiment, the modified release component may comprise about 40% or less of the total amphetamines in the tablet, based on the weight of the free amphetamine base, and further wherein the immediate release component provides about 60% or more of the total amphetamines in the tablet, wherein the weight is measured on the basis of free amphetamine base. In one embodiment, a chewable tablet as provided herein is further characterized by also being an orally disintegrating tablet.

Still other aspects and advantages of the invention will be apparent from the following detailed description of the invention.

### BRIEF DESCRIPTION OF THE DRAWINGS

FIG. 1 provides a graph of the plasma concentration profile for d-amphetamine following administration of a chewed 20 mg tablet under fasted conditions (A), a chewed tablet under fed conditions (B) or a tablet swallowed whole (C), as compared to a liquid ER suspension product (line D).

FIG. 2 provides a graph of the plasma concentration profile for l-amphetamine following administration of a chewed 20 mg tablet under fasted conditions (A), a chewed tablet under fed conditions (B) or a tablet swallowed whole (C), as compared to a liquid ER suspension product (line D).

FIG. 3 provides a boxplot for Time to Collision (TOC) Mean Min in the within-subjects comparisons of driving variables in the study described in Example 15, relating to a randomized, double-blind, placebo-controlled study using a laboratory driving environment in young adult drivers with ADHD. The subjects were provided with a single dose of an amphetamine extended-release tablet as described herein. See, also Table in Example 15 with descriptive statistics associated with the within-subjects comparisons.

FIG. 4 provides a boxplot for TOC Median Min in the within-subjects comparisons of driving variables in the study described in Example 15, relating to a randomized, double-blind, placebo-controlled study using a laboratory driving environment in young adult drivers with ADHD. The subjects were provided with a single dose of an amphetamine extended-release tablet as described herein. See, also Table in Example 15 with descriptive statistics associated with the within-subjects comparisons.

FIG. 5 provides a boxplot for TOC Min in the within-subjects comparisons of driving variables in the study described in Example 15, relating to a randomized, double-blind, placebo-controlled study using a laboratory driving environment in young adult drivers with ADHD. The subjects were provided with a single dose of an amphetamine extended-release tablet as described herein. See, also Table in Example 15 with descriptive statistics associated with the within-subjects comparisons.

FIG. 6 provides a boxplot for Braking Percent in the within-subjects comparisons of driving variables in the study described in Example 15, relating to a randomized, double-blind, placebo-controlled study using a laboratory driving environment in young adult drivers with ADHD. The subjects were provided with a single dose of an amphetamine extended-release tablet as described herein. See, also Table in Example 15 with descriptive statistics associated with the within-subjects comparisons.

FIG. 7 provides a boxplot for Mean Max Brake Force in the within-subjects comparisons of driving variables in the study described in Example 15, relating to a randomized, double-blind, placebo-controlled study using a laboratory driving environment in young adult drivers with ADHD. The subjects were provided with a single dose of an amphetamine extended-release tablet as described herein. See, also Table in Example 15 with descriptive statistics associated with the within-subjects comparisons.

FIG. 8 provides a boxplot for Mean Max Brake Force Zeroes in the within-subjects comparisons of driving variables in the study described in Example 15, relating to a randomized, double-blind, placebo-controlled study using a laboratory driving environment in young adult drivers with ADHD. The subjects were provided with a single dose of an amphetamine extended-release tablet as described herein. See, also Table in Example 15 with descriptive statistics associated with the within-subjects comparisons.

FIG. 9 provides a boxplot for Mean Throttle Release Reaction Time (RT) in the within-subjects comparisons of driving variables in the study described in Example 15, relating to a randomized, double-blind, placebo-controlled study using a laboratory driving environment in young adult drivers with ADHD. The subjects were provided with a single dose of an amphetamine extended-release tablet as described herein. See, also Table in Example 15 with descriptive statistics associated with the within-subjects comparisons.

FIG. 10 provides a boxplot for Mean Brake RT Zeros in the within-subjects comparisons of driving variables in the study described in Example 15, relating to a randomized, double-blind, placebo-controlled study using a laboratory driving environment in young adult drivers with ADHD. The subjects were provided with a single dose of an amphetamine extended-release tablet as described herein. See, also Table in Example 15 with descriptive statistics associated with the within-subjects comparisons.

FIG. 11 provides a boxplot for Mean Velocity in the within-subjects comparisons of driving variables in the study described in Example 15, relating to a randomized, double-blind, placebo-controlled study using a laboratory driving environment in young adult drivers with ADHD. The subjects were provided with a single dose of an amphetamine extended-release tablet as described herein. See, also Table in Example 15 with descriptive statistics associated with the within-subjects comparisons.

FIG. 12 provides a boxplot for Mean Standard Deviation (SD) Velocity in the within-subjects comparisons of driving variables in the study described in Example 15, relating to a randomized, double-blind, placebo-controlled study using a laboratory driving environment in young adult drivers with ADHD. The subjects were provided with a single dose of an amphetamine extended-release tablet as described herein. See, also Table in Example 15 with descriptive statistics associated with the within-subjects comparisons.

FIG. 13 provides a boxplot for Mean Standard Deviation of Lane Position (SDLP) in the within-subjects comparisons of driving variables in the study described in Example 15, relating to a randomized, double-blind, placebo-controlled study using a laboratory driving environment in young adult drivers with ADHD. The subjects were provided with a single dose of an amphetamine extended-release tablet as described herein. See, also Table in Example 15 with descriptive statistics associated with the within-subjects comparisons.

FIG. 14 provides a boxplot for Mean Small Steering Wheel Reversals (SWR) in the within-subjects comparisons of driving variables in the study described in Example 15, relating to a randomized, double-blind, placebo-controlled study using a laboratory driving environment in young adult drivers with ADHD. The subjects were provided with a single dose of an amphetamine extended-release tablet as described herein. See, also Table in Example 15 with descriptive statistics associated with the within-subjects comparisons.

FIG. 15 provides a boxplot for Mean Lane Excursion Count in the within-subjects comparisons of driving variables in the study described in Example 15, relating to a randomized, double-blind, placebo-controlled study using a laboratory driving environment in young adult drivers with ADHD. The subjects were provided with a single dose of an amphetamine extended-release tablet as described herein. See, also Table in Example 15 with descriptive statistics associated with the within-subjects comparisons.

### DETAILED DESCRIPTION OF THE INVENTION

Methods are provided herein for improving driving behavior and reducing traffic accidents in a human subject operating a motor vehicle. In certain embodiments, the subjects have attention deficit hyperactivity disorder (ADHD) or a related condition (e.g., attention deficit disorder). In certain embodiments, the method involves dosing the subject once daily with an amphetamine extended release tablet provided herein. In certain embodiments, a method for decreasing the risk of a driving accident in a human operating a motor vehicle is provided. In certain embodiments, the method involves providing the subject with an amphetamine extended release tablet composition as provided herein prior to beginning. In certain embodiments, improvements in driving behavior are obtained within about 45 minutes post-dosing and through about 10 hours post-dosing. In certain embodiments, improvements may be observed in less than 45 minutes and for longer than 10 hours post-dosing. In certain embodiments, a human subject is dosed at least 30 minutes prior to operating a motor vehicle. In other embodiments, a human subject is dosed at least 45 minutes prior to operating a motor vehicle. In certain embodiments, a human subject operates the motor vehicle within 10 hours post-dosing. However, later time periods may be selected.

A “motor vehicle” as used herein includes a land-based vehicle such as an automobile (car), bus, motorcycle, scooter, off-road vehicle, light truck and/or a regular truck. In certain embodiments, the methods provided herein may be used for operators of trains (e.g., light rail, heavy rail, or the like), or watercraft (e.g., boat, ships, etc).

In certain embodiments, the subject is 16 to 100 years old, or ages in between, e.g. 18 to 90 years old, 21 to 85 years old, 16 to 60 years old, 18 to 25 years old, 18 to 60 years old, 18 to 40 years old, or ages in between. Suitably, the subject is trained and/or licensed, or has a valid permit, to operate a motor vehicle.

As used herein, “improving driving skills”, may include any suitable factor, such as, without limitation, improved reaction time, decreased collision(s), decreases in deviation of lane position and/or lane excursions, decreased steering wheel deviations, improved reaction time to throttle release, improved brake force, improved rapid velocity, improved time to breaking.

Useful in the methods provided herein are amphetamine extended release compositions in which at least two different forms of amphetamine are provided in different release profiles, i.e., a modified release amphetamine component and an immediate release amphetamine component. In one embodiment, there is more than one immediate release component, one of which has a faster onset, e.g., up to about 100% of this faster onset is released within about 10 to about 20 minutes, and a second immediate release component, e.g., up to about 100% is released within about 1 hour. In one embodiment, at least two different amphetamine compounds (e.g., racemic amphetamine, d-amphetamine, l-amphetamine, mixtures thereof, or salts thereof) are present in each of the modified release and immediate release profiles.

As used herein, the term “amphetamine”, abbreviated herein “AMP” is used to refer generally to amphetamine compounds, encompassing racemic amphetamine and the separate enantiomers thereof: dextro- or dex- (d)-amphetamine, and/or levo (l)-amphetamine. Racemic amphetamine is also termed herein (d,l)-amphetamine and is generally understood to contain a 1:1 ratio of d- to l-amphetamine. However, a racemate produced containing other ratios of these enantiomers may be used in the preparing the compositions described herein. Methods of preparing optically pure amphetamine enantiomers have been described. See, e.g., U.S. Pat. Nos. 3,028,430; 2,906,665A (l-amphetamine alginate); U.S. Pat. No. 6,399,828 (preparing amphetamines from phenylpropalamines); U.S. Pat. No. 8,487,134B2 (production acylated amphetamine, dexamphetamine and methamphetamines), the disclosures of which are incorporated by reference. Various amphetamine active pharmaceutical ingredients (API)-grade compounds can be purchased commercially (e.g., Gyma Laboratories of America, Cambrex, Johnson Matthey). Typically, these compounds are purchased in the form of a pharmaceutically acceptable salt thereof, such as the mesylate, hydrochloride, saccharate, sulfate, aspartate salt, or a hydrate of such a salt. These or other pharmaceutically acceptable salts include e.g., non-toxic, inorganic and organic acid addition salts, are known in the art. Exemplary salts include, but are not limited to, 2-hydroxyethanesulfonate, 2-naphthalenesulfonate, 3-hydroxy-2-naphthoate, 3-phenylpropionate, acetate, adipate, alginate, amsonate, aspartate, benzenesulfonate, benzoate, besylate, bicarbonate, bisulfate, bitartrate, borate, butyrate, calcium edetate, camphorate, camphorsulfonate, camsylate, carbonate, citrate, clavulariate, cycloperitanepropionate, digluconate, dodecylsulfate, edetate, edisylate, estolate, esylate, ethanesulfonate, fumarate, gluceptate, glueoheptanoate, gluconate, glutarnate, glycerophosphate, glycollylarsanilate, hemisulfate, heptanoate, hexafluorophosphate, hexanoate, hexylresorcinate, hydrabamine, hydrobromide, hydrochloride, hydroiodide, hydroxynaphthoate, iodide, isothionate, lactate, lactobionate, laurate, laurylsulphonate, malate, maleate, mandelate, mesylate, methanesulfonate, methylbromide, methylnitrate, methylsulfate, mucate, naphthylate, napsylate, nicotinate, nitrate, N-methylglucamine ammonium salt, oleate, oxalate, palmitate, pamoate, pantothenate, pectinate, persulfate, phosphate, phosphate/diphosphate, picrate, pivalate, polygalacturonate, propionate, p-toluenesulfonate, saccharate, salicylate, stearate, subacetate, succinate, sulfate, sulfosalicylate, suramate, tannate, tartrate, teoclate, thiocyanate, tosylate, triethiodide, undecanoate, and valerate salts, and the like. In one embodiment, in the compositions used in the methods described herein, the saccharate salt form of the amphetamine is not selected for use in a composition where the counterion will be present in the composition.

As used herein, a “counterion” is the ion that accompanies an ionic species (e.g., an amphetamine base) in order to maintain electric neutrality. For example, when an amphetamine salt is dissolved, the “counterion” is the dissociated ionic species, e.g., from an amphetamine saccharate, the saccharate would be the counterion.

Although not encompassed with the term “amphetamine”, it will be understood that derivatives, prodrugs (e.g., esters), and/or pre-prodrugs of the racemic amphetamine, dex(d)-amphetamine, or l-amphetamine, may be selected for use in a composition described herein.

One example of such a drug includes, e.g., lisdexamphetamine. Methods for producing such derivatives, prodrugs and pre-prodrugs have been described. See, e.g., U.S. Pat. No. 8,487,134 (production of acylated amphetamine, dexamphetamine and methamphetamines); US20120157706 (methods and compositions for preparing lisdexamfetamine and salts thereof), the disclosures of which are incorporated herein by reference.

In one embodiment, the compositions used as provided herein are contain a total ratio of d- to l-amphetamine of about 3 parts by weight d-amphetamine to about 1 part by weight l-amphetamine (e.g, 2.60 parts by weight to 3.40 parts by weight d-amphetamine, or 3 parts by weight to 3.4 parts by weight d-amphetamine, or about 3.2 parts by weight d-amphetamine) to about 1 part by weight l-amphetamine. Further, the modified release and/or one or both of the immediate release components may also contain a similar ratio. When making this calculation, the total d-amphetamine amount is used, regardless of whether contributed by the racemate or the dexamphetamine enantiomer.

Unless otherwise specified, the racemate contains 50% d- and 50% l-amphetamine. Throughout this specification, when weight percentages and/or ratios are provided for amphetamines in each of the active components, the weight percentage or ratio is calculated based on the amount of amphetamine base in each component.

As used herein the term “uncomplexed” refers to an amphetamine, refers to the faster onset immediate release component, wherein a second immediate onset component is present, and specifically includes the corresponding amphetamine free base, as well as pharmacologically active and physiologically compatible salts form thereof, including acid addition salts, hydrates thereof, and hydrates (anhydrous, semihydrates, etc) of these salts; specifically excluded from the term “uncomplexed amphetamine” is an amphetamine which bound to or complexed with a cation exchange resin.

The immediate release amphetamine component also comprises an amphetamine-cation exchange resin complex, optionally in combination with a matrix forming polymer which is characterized herein as the “slower” onset immediate release component. This component may be referred to as “uncoated” in that it may contain no coating, or if any coating is present, the coating either does not function to modify the release characteristics of the drug (from immediate release to modified release). This slower onset immediate release component may contain a blend of two separate uncoated amphetamine-cation exchange resin complexes, each of which contains a single amphetamine component bound to a cation exchange resin (e.g., (d,l)-amphetamine-cation exchange resin complex and/or d-amphetamine-cation exchange resin complex). Alternatively, two different amphetamines may be bound to a single cation exchange resin complex (e.g., both (d,l)- and d-amphetamine-cation exchange resin complex). Methods for loading multiple drugs onto a single resin have been described. See, e.g., U.S. Pat. Nos. 8,062,667, 8,329,224 B2 and Published Patent Application No. 2007-0148239, which describes multiple loadings onto an ion exchange resin; and WO 2007/001300, which generally describes drug loading and conjugating more than one active drug with a single resin particle, the disclosures of which are incorporated by reference herein. In still another embodiment, compositions may be produced in which amphetamine-cation exchange resin complexes containing multiple amphetamines (e.g., dl-,d-amphetamine-cation exchange resin complex) and containing a single amphetamine (d-amphetamine-cation exchange resin complex) are combined. The immediate release component contains a plurality of particulate, amphetamine-cation exchange resin complexes.

As used herein, the term “modified release” refers to components in which the release of at least one of the active components (i.e., (d,l)-amphetamine, d-amphetamine or l-amphetamine) is longer than its immediate release form. “Modified release” may encompass sustained release, extended release, or delayed release. For convenience, “modified release” is used to refer to the amphetamine component of the composition, whereas “extended release” is used to refer to the present composition which provides both immediate release and modified release components. In one embodiment, the modified release profile extends to at least 8 hours. More desirably, the composition provides a therapeutic effect through at least about 12 hours to about 18 hours, about 13 hours to about 16 hours, or about 13 hours. The release profile may be assessed via in vitro dissolution using techniques known to those of skill in the art [e.g., USP basket method, paddle method, channel flow method, or other methods known in the literature]. The release profile can be assessed in vivo (e.g., for bioavailability determinations), using plasma concentrations to assess maximum plasma concentration (Cₘₐₓ) and area under the curve (AUC).

A therapeutic result for amphetamine is not solely related to plasma levels of the drug. “A therapeutically effective amount” of amphetamine includes the minimum amount of the drug required to provide a clinically observable psychological and/or behavioral response. Unless otherwise specified, this is determined following a single or “initial” administration of an extended release amphetamine composition as described herein.

The term “initial administration” is defined for purposes of the present invention as the first single dose of an extended release amphetamine composition administered to a patient or subject or the first dose administered to a patient or subject after a suitable washout period.

As used herein, the term “patient” and “subject” are used interchangeably to refer to humans who are being dosed with an extended release amphetamine composition as provided herein.

The modified release component has a coating which provides a modified release profile to the amphetamine(s). The coating is over the amphetamine-cation exchange resin complex, which is optionally in a matrix. As described in more detail below, the modified release component may contain a blend of two different modified release, pH-independent, barrier coated-amphetamine-cation exchange resin complexes, each of which contains a single amphetamine component bound to a cation exchange resin. Alternatively, at least two different amphetamines may be bound to a single cation exchange resin complex (e.g., (d,l)-amphetamine, d-amphetamine, and/or l-amphetamine), using methods such as are described herein. In still another embodiment, compositions may be produced in which two or more different amphetamines on the same cation exchange resin is combined with a single amphetamine bound to a separate cation exchange. The resulting modified release component contains a plurality of particulate, barrier coated amphetamine-cation exchange resin complexes, which are separately coated with a pH-independent release, high tensile strength, water insoluble, water-permeable modified release barrier coating prior to be admixed with the immediate release components. In the compositions described herein, typically, the excipients are selected such that they do not contribute at all, or to any measurable amount, to the modified release properties of the composition.

Whether selected from the racemate (d,l), or the separate enantiomers: d-amphetamine or l-amphetamine, the active drug may be present in the form of a salt, hydrate, or hydrate of a salt. One suitable salt is the saccharate, succinate, hydrochloride, aspartate or sulfate salt form. However, other pharmaceutically acceptable salts may be selected.

As used herein, the term “free amphetamine” or “free amphetamine base” refers to the weight of the amphetamine base, i.e., exclusive of any salt, hydrate, polistirex or complex form (i.e., without the counterion, any water content, or and/or ion exchange resin).

In one embodiment, an amphetamine extended release chewable tablet useful in the methods provided herein contains amphetamines in different forms, (a) a modified release barrier coated amphetamine-cation exchange resin complex, optionally in a matrix, (b) a slower onset immediate release uncoated amphetamine-cation exchange resin complex, optionally in a matrix, and (c) a faster onset immediate release uncomplexed amphetamine. The modified release, barrier coated amphetamine-cation exchange resin complex, optionally in a matrix, may contain a mixture of one or more of (d,l)-amphetamine, d-amphetamine and/or l-amphetamine complexed on separate ion exchange resins, or a mixture of d-amphetamine and l-amphetamine complexed onto the same cation exchange resin complex.

The compositions provided herein are characterized by providing both immediate release amphetamine and modified release amphetamine. The term “immediate release” (“IR”) refers to the release of an active ingredient (e.g., an amphetamine) from a pharmaceutical formulation where the rate of release of the active pharmaceutical ingredient from the pharmaceutical formulation is not substantially retarded by means of a controlled release matrix or other such means and where the components of the pharmaceutical formulation are designed such that, upon ingestion, maximum exposure of said active pharmaceutical ingredient to body tissues occurs in the minimum period of time. As described herein, an “immediate release” amphetamine component releases about 100% in less than 1 hour. The present invention provides for an extended release composition having two different immediate release amphetamine components, each of which provides a different release profile.

In one embodiment, the composition contains at least a first, faster onset immediate release component, i.e., release of about 100% in less than 30 minutes, with at least 50% release in less than 20 minutes and, preferably in as few as ten minutes, or sooner. In some embodiments, 80 to 100% is released within about 10 to 20 minutes, or about 10 minutes. This faster onset immediate release component comprises at least one uncomplexed amphetamine. A second, different, slower onset immediate release component provides a different immediate release pharmacokinetic profile, which releases in less than about an hour, as soon as about 30 minutes, or as soon as about 20 minutes, with at least some measurable release as early as about 10 to 15 minutes. Suitably this slower onset immediate release component is an uncoated amphetamine-cation exchange resin complex, which is optionally in a matrix with a matrix forming polymer. When present in the immediate release component, the matrix forming polymer is selected so that the resulting uncoated amphetamine-cation exchange resin complex (optionally in a matrix) retains an immediate release profile. For convenience, the optional matrix is not referenced in every phrase where the uncoated complex is discussed. However, it will be understood that this uncoated complex may contain such a component. The release profiles of the two different immediate release components may overlap, e.g., the slower onset immediate release component may begin releasing before 100% of the faster onset immediate release component is completely released.

In certain embodiments, the modified release barrier coated amphetamine-cation exchange resin complex, optionally in a matrix provides an amount of about 10% w/w to about 40% w/w, about 15% w/w, about 20% w/w, about 25% w/w, about 30% w/w, or about 40% w/w of the total amphetamine components in the composition (based on the free amphetamine base(s)). The two immediate release components combine to provide about 60% w/w to about 90% w/w, or about 70%, or about 75%, or about 80%, or about 85% w/w, or values there between of the total amphetamine components in the composition. The uncoated amphetamine-cation exchange resin complex component is designed to be immediate release as defined herein, and as such, does not contain a coating which functions to delay release (e.g., no functional amount of an extended release barrier coating or enteric coat). Suitably, immediate release amphetamine-cation exchange resin complex is present in an amount of about 20% w/w to about 75% w/w, or about 25% w/w to about 70% w/w, about 50% w/w, about 55% w/w, about 60% w/w, about 70% w/w, about 75% w/w of the amphetamine components in the composition based on the total amount of free amphetamine base.

The extended release component and the immediate release amphetamine-cation exchange resin complex are further in combination with an uncomplexed amphetamine drug(s). The other immediate release component, which is an uncomplexed amphetamine drug, is present in an amount of about 5% w/w to about 25% w/w, or about 15% w/w to about 25% w/w, or about 20% w/w of the amphetamine components in the compositions based on the total amount of free amphetamine base. In one embodiment, a composition contains a ratio of amphetamine in a coated amphetamine-cation exchange resin complex: uncoated amphetamine-cation exchange resin complex: uncomplexed amphetamine is about 20 to about 30: about 50 to about 60: about 15 to about 25. In one embodiment, the weight percentages of amphetamine contributed by each of the two immediate release components are the same. However, in other embodiments, it may be desirable to provide amphetamine in the immediate release components in different weight percentages.

An “amphetamine-cation exchange resin complex” refers to the product resulting from loading an amphetamine which has been disassociated with any counterion and bound to a cation exchange resin.

Methods for preparing such complexes have been described, e.g., in WO 2007/109104, the disclosure of which is incorporated herein by reference. This describes the complexation which occurs when the active drug and the cation exchange resin are mixed together in an aqueous medium to facilitate the “exchange” between the salt of the amphetamine and the “cation” of the cation exchange resin and the formation of the complex, which may be referred to as “amphetamine polistirex”. WO 2007/109104 also describes polyvinylacetate-based barrier coatings which are particularly well suited for use in the formulations described herein to provide a modified release coat over the amphetamine-cation exchange resin complex-matrix. However, one skilled in the art can select other barrier coatings to provide the modified release characteristics to amphetamine-cation exchange resin complex-matrix.

The term “amphetamine-cation exchange resin complex” and “amphetamine polistirex” are used interchangeably throughout this specification. Further, the term “amphetamine-cation exchange resin complex” or “amphetamine polistirex” includes when more than one form of an amphetamine is complexed to the same cation exchange resin, e.g., a cation exchange resin may have both racemic (d,l)-amphetamine and d-amphetamine complexed thereto.

As used herein, a “precoated” amphetamine-cation exchange resin complex or a “precoated” amphetamine-cation exchange resin complex-matrix refers to a particle which is to be subsequently coated with a barrier coating as defined herein. In some embodiments, where the amphetamine-cation exchange resin or amphetamine-cation exchange resin complex-matrix is to be used as one of the immediate release components and no barrier coating is to be applied, it is referred to as “uncoated”. In some embodiments, the “immediate release coated amphetamine-cation exchange resin complex” may have a selected coating thereon, where the coating does not confer any modified release to the amphetamine.

As used herein, a barrier coat is a water-permeable, water-insoluble, pH-independent polymer or co-polymer which in the present invention confers modified release to the coated amphetamine-cation exchange resin complex-matrix. In one embodiment, the barrier coat is pH-independent, non-ionic and is applied, e.g., as an aqueous suspension, over the precoated amphetamine-cation exchange resin complex-matrix and forms a separate layer thereon. In another embodiment, the barrier coat is pH-independent, non-ionic and is applied as a solvent-based coating, over the amphetamine-cation exchange resin complex-matrix and forms a separate layer thereon. In still another embodiment, the barrier coat is pH-independent, ionic and is applied over the amphetamine-cation exchange resin complex-matrix to form a separate layer thereon. Preferably, the barrier coat is directly over the amphetamine-cation exchange resin complex-matrix and the barrier coat layer, i.e., there are no intervening layers between the barrier coat and the amphetamine-cation exchange resin complex-matrix. Depending upon the polymeric material selected, the barrier coat polymer or co-polymer may be cured to maximize its properties. These polymers and their curing requirements are discussed in more detail elsewhere in this specification.

An “amphetamine-cation exchange resin complex-matrix” refers to an amphetamine-cation exchange resin complex which is further combined, e.g., prior to or during granulation, with a polymeric material which forms a matrix with the amphetamine-cation exchange resin complex.

The term “matrix forming polymer” or “matrix forming polymeric material” refers to both water-insoluble (hydrophobic) polymers and co-polymers and water-soluble (hydrophilic) polymers and co-polymers which form a matrix with the amphetamine-cation exchange resin complex upon being admixed or granulated therewith. Suitably, the matrix forming polymer is non-reactive with the AMP and the cation exchange resin. The matrix forming polymer may be a water-insoluble polymers/co-polymers and polymer systems which have been described as release retardants [see, e.g., polymers discussed in U.S. Pat. No. 8,062,677, the disclosure of which is incorporated herein by reference], and those hydrophilic polymer systems which have been described in the literature as impregnating or solvating agents [see, e.g., polymers discussed in U.S. Pat. Nos. 8,062,677 and 4,221,778, incorporated herein by reference]. In one embodiment, an amphetamine-cation exchange resin complex-matrix may include more than one matrix-forming polymer system. For example, an amphetamine-cation exchange resin complex-matrix may contain both a hydrophilic polymer and a hydrophobic polymer.

An immediate release “uncoated amphetamine-cation exchange resin complex” may optionally be in a matrix. In this instance, the matrix forming polymer does not substantially alter the ability of the amphetamine-cation exchange resin complex-matrix to provide an immediate release profile. For example, a polyvinylpyrrolidone may be selected as a matrix forming polymer. Alternatively, the matrix forming polymer may alter the release rate of this complex while still maintaining an immediate release profile as defined herein.

The following terms are used in the specification and are to be interpreted in accordance with the definitions herein.

“Cₘₐₓ” is the maximum plasma concentration, calculated as the geometric or arithmetic mean of the individual maximum blood plasma concentrations over the sampling period.

The term “Tₘₐₓ” is the time at which the peak (maximum) observed blood plasma drug concentration for each individual participating in the bioavailability study over the sampling period.

The term “AUC_{0-∞}” or “AUC_{inf}” is the mean area under the analyte (e.g., from plasma or serum) concentration-time curve extrapolated to infinity. It is calculated as the mean of the area under the analyte concentration-time curve from time 0 extrapolated to infinity, calculated for each individual participating in the bioavailability study and may be the geometric or arithmetic mean. In certain embodiments, AUC0-∞=AUC0-t+Ct/kel, where Ct is the last measurable analyte concentration and kel is the apparent first-order elimination rate constant.

AUC₀₋ₜ is the area under the plasma/serum/blood concentration-time curve from time zero to time t, where t is the last time point with measurable analyte concentration. t may be, e.g., 1 hour (AUC₀₋₁), 2 hours (AUC₀₋₂), 3 hours (AUC₀₋₃), 4 hours (AUC₀₋₄), 5 hours (AUC₀₋₅), or at 0-4 hours, 4-8 hours, 8-12 hours, 8-13 hours, under fasting and/or fed conditions, or at different intervals, including those where t is 13 hours, 14 hours, 15 hours, 16 hours, 17 hours, 18 hours, or up to 24 hours as end time points.

Partial AUC may be useful in determining bioequivalence, where the AUC is determined based on a specific fragment of the AUCₜ₋ₜ₊ₓ. These fragments may be, e.g., from 0-2 hours, 2-4 hours, 5-8 hours, 8-13 hours, or other time points.

In certain embodiments, the AUC values are calculated by the linear trapezoidal rule.

“Bioequivalent” means the pharmacokinetic profile of a test composition is within the range of about 80% to about 125%, when compared to the values of one or more of the AUC values or the Cₘₐₓ of the reference composition. See, e.g., US Department of Health and Human Services. Guidance for Industry: Bioavailability and Bioequivalence Studies for Orally Administered Drug Products—General Considerations. Washington, DC: Center for Drug Evaluation and Research; March 2003. For example, in certain embodiments, a bioequivalent product is required to be within 80% to 125% of AUC_{0-∞}, or within 90% to 110% of AUC_{0-∞}. In certain embodiments, a bioequivalent product is bioequivalent over preselected partial AUC values. For example, in certain embodiments, being bioequivalent to the AUC₀₋₂ values of the composition of the invention is desired. Typically, in order to be bioequivalent, a product must match not just a single partial AUC, but also either be bioequivalent over the entire time period and/or other partial AUC time periods In order to declare the absence of a food effect on the test formulation, the 90% CIs of the relative mean plasma d-amphetamine and l-amphetamine of one or more of AUC₀₋₂, AUC₀₋₅, AUC₅₋ₜ, AUC_{0-∞}, and/or Cₘₐₓ of the test product under fed versus fasted conditions should be between 80.00 and 125.00%, inclusive. See, e.g., US Department of Health and Human Services. Guidance for Industry: Food Effect Bioavailability and Fed Bioequivalence Studies. Rockville, MD: Center for Drug Evaluation and Research; December 2002. In one embodiment, a composition of the invention has no food effect, i.e. there is no statistical difference in one or more of the pharmacokinetic parameters, including, e.g., Cmax, AUC0-∞″, and/or one or more partial AUCs, when patients are administered a dose under fasted conditions as compared to fed conditions.

In order to establish relative bioavailability of a test formulation (e.g., administered as a whole tablet to the reference or chewing under fasted conditions), the 90% confidence intervals (CIs) of the ratios of geometric mean plasma d-amphetamine and l-amphetamine of one or more of AUC₀₋₂, AUC₀₋₅, AUC₅₋ₜ, AUC_{0-∞}, and/or Cₘₐₓ, of the test product in relation to the reference product should be between 80.00 and 125.00%, inclusive. See, e.g., US Department of Health and Human Services. Guidance for Industry: Bioavailability and Bioequivalence Studies for Orally Administered Drug Products—General Considerations. Washington, DC: Center for Drug Evaluation and Research; March 2003.

When reference is made to “different amphetamines”, it will be understood that a racemic amphetamine, a d-amphetamine, and an l-amphetamine are considered herein to be “different” from one another. As a whole, they may be considered “amphetamines”. Similarly, the other types of amphetamines or amphetamine derivatives as defined herein are encompassed by this definition. In some circumstances, counterions which form salts with these free bases are referred to as “different counterions”. This will be readily understood to refer to the counterions of a different free radical which is capable of forming a pharmaceutically acceptable salt with an amphetamine free base.

The term “half-life” is the apparent terminal elimination half-life (T_{1/2}).

The words “comprise”, “comprises”, and “comprising”, and “contain”, “containing”, and “contains” are to be interpreted inclusively rather than exclusively. The words “consist”, “consisting”, and its variants, are to be interpreted exclusively, rather than inclusively.

As used herein in reference to numeric values provided herein, the term “about” may indicate a variability of as much as 10% (up to ±10% of the indicated value, including). This includes lower variabilities (e.g., ±1%, ±2%, ±3%, ±4%, ±5%, ±6%, ±7%, ±8%, ±9%, and other values between 0 and 10).

### Amphetamine-Cation Exchange Resin Complexes

As described herein, a polistirex or drug-cation exchange resin may contain one form of an amphetamine, e.g., racemic (d,l) amphetamine, or two or more amphetamine compounds. An amphetamine can be complexed with, or loaded onto, a cation exchange resin, using methods which are known in the art. See, e.g., WO 2007/109104, the disclosure of which is incorporated by reference, and the documents cited therein. One or more different amphetamine compounds [e.g., racemic, d-, or l-amphetamine] may be synthesized or obtained from commercial sources as described above.

Cationic exchange resins are readily selected for use as described herein. For example, cationic exchange resins vary in strength, i.e., in their ability to exchange cations. In one embodiment, a relatively strong cationic resin, e.g., Amberlite® IRP69, manufactured by Dow Chemical (a sulfonated copolymer of styrene and divinylbenzene) is selected. Alternatively, one may select a relatively weak cationic exchange resin, e.g., Amberlite® IRP88 [Dow Chemical (which acquired previous manufacturer Rohm & Haas) a crosslinked polymer of methacrylic acid and divinylbenzene)], a weakly acidic (potassium ion) cation exchange resin with 4% cross-linked methacrylate (100 to 500 mesh, equivalent to about 150 microns to about 27 microns, ASTM standard) or Amberlite® IRP64 (a methacrylic acid and divinylbenzene polymer (hydrogen ion) polacrilex resin, Rohm and Haas, with a particle size ranging from 100 to 400 mesh (equiv to 35 microns to 150 microns, ASTM standard size), capacity ˜10 meq/g by dry weight). Further, either regularly or irregularly shaped particles may be used as cation exchange resins according to the present invention. Regularly shaped particles are those particles that substantially conform to geometric shapes such as spherical, elliptical, cylindrical and the like, which are exemplified by Dowex® 50WX8 (The Dow Chemical Company). Irregularly shaped particles are all particles not considered to be regularly shaped, such as particles with amorphous shapes and particles with increased surface areas due to surface channels or distortions. Irregularly shaped ion-exchange resins of this type are exemplified by Amberlite® IRP-69 (manufactured by Dow Chemical), the use of which is illustrated in the examples below. This cation exchange resin is a sulfonated polymer composed of polystyrene cross-linked with about 8% of divinylbenzene, with an ion-exchange capacity of about 4.5 to 5.5 meq/g of dry resin (H⁺-form). Another cation exchange resin having similar properties is Dowex® 50WX8 (H+ form, linear formula, C₁₀H₁₂·C₁₀H₁₀·C₈H₈)ₓ, 200-400 mesh particle size, which is equivalent to about 75 microns to about 35 microns, ASTM standard). Amberlite® IRP-69 consists of irregularly shaped particles with a size range of about 100 to about 500 mesh (about 150 microns to about 27 microns, ASTM standard). Dowex® 50WX8 is more regularly shaped. Resins are generally purchased with a size ranging from about 25 microns to about 400 microns. However, other sizes may be selected, or larger sized particles may be milled to provide smaller particle sizes.

The selected ion-exchange resins may be further treated by the manufacturer or the purchaser to maximize the safety for pharmaceutical use or for improved performance of the compositions. Impurities present in the resins may be removed or neutralized by the use of common chelating agents, anti-oxidants, preservatives such as disodium edetate, sodium bisulfite, sodium metabisulfite, and so on by incorporating them at any stage of preparation either before complexation or during complexation or thereafter. These impurities along with their chelating agent to which they become bound may be removed before further use of the cation exchange resin.

A method for preparing a drug-cation exchange resin complex involves reacting the drug(s) and cation exchange resin in water under conditions which permit formation of a complex. Typically, the reaction step involves combining the active drug(s) or a pharmaceutically acceptable salt thereof and a pharmaceutically acceptable, water-insoluble, cation exchange resin in an aqueous admixture and mixing for a sufficient amount of time to allow binding of the drug free base to the cation exchange resin. Optionally, a single amphetamine drug may be used in the following methods in a single complexing (loading) step to form an amphetamine-cation exchange resin complex containing a single amphetamine drug (e.g., d-amphetamine). These steps may be repeated in separate processes for the other amphetamine-cation exchange resin complexes to be used in a composition of the invention. Alternatively, a mixture of amphetamine drugs may be used in a single complexing step to provide an amphetamine-cation exchange resin complex which contains a mixture of amphetamine drugs (e.g., (d,l)-amphetamine, d-amphetamine, and/or l-amphetamine). Mixing during the complexing step may be, e.g., for about 1 hour to about 5 hours, or about 2 hours, followed by further processing which may take up to about 72 hours or longer.

The order of admixing in the complexation step; the drug(s) and the cation exchange resin may be performed in multiple ways. Optionally, a cation exchange resin for preparing a complex may be pre-washed, e.g., according to its manufacturer's instructions or as previously described. See, e.g., U.S. Pat. No. 8,062,667, the disclosure of which is incorporated by reference herein.

As described above, the reaction between the drug (e.g., (d,l)-amphetamine and/or d-amphetamine) and cation exchange resin occurs in a suspension having a water content in excess of 50% w/w, more typically in the range of about 60% to about 90% w/w to facilitate complexing of the amphetamine with the cation exchange resin. While the moisture content may be provided by water alone, optionally, solvents in addition to water may be present in this aqueous reaction slurry. For example, suitable solvents may include isopropyl alcohol, methanol or ethanol, or mixtures thereof in the aqueous slurry. In certain embodiments, the moisture content of the slurry may be reduced to below about 50% w/w, and more desirably, in the range of about 20% w/w to about 30% w/wand/or, more preferably 2 to 10% w/w. During this processing, all or substantially any remaining counter-ion of the starting pharmaceutically active salt of the drug is removed through the washing steps.

The total amount of amphetamine that can be complexed with a resin will typically range from about 5% to about 50% by weight of the amphetamine-cation exchange resin complex particles. A skilled artisan with limited experimentation can determine the optimum loading for any amphetamine-cation exchange resin complex. In one embodiment, loading of about 10% to about 40% by weight, more desirably, about 15% to about 30% by weight, or about 25% of the amphetamine-cation exchange resin complex particles can be employed. In one embodiment, a composition of the invention contains amphetamine complexed to a sodium polystyrene sulfonate resin in at a ratio of 20 parts amphetamine (based on the weight of the amphetamine salt) to 300 parts cation exchange resin or 80 parts amphetamine (based on the weight of the amphetamine salt) to 100 parts cation exchange resin. In another embodiment, the amphetamine (based on the weight of the amphetamine salt) to resin ratio is 4:1 to 1:15, or 4:1 to about 1:4, or about 1:1. In a further embodiment, the amphetamine-cation exchange resin may contain more than one amphetamine compound bound to the same cation exchange resin, e.g., (d,l)-amphetamine and d-amphetamine. In certain embodiments, both the modified release component and the immediate release amphetamine-cation exchange resin complex contain two different amphetamines bound to the same cation exchange resin complex. In another embodiment, one of the immediate release components contains a blend of amphetamine-cation exchange resin complexes, in which a (d,l)-amphetamine-cation resin complex and a d-amphetamine-cation exchange resin complex are blended or admixed. In another embodiment, the modified release component contains a blend of barrier coated amphetamine-cation exchange resin complex-(optional matrix), in which a modified release, barrier coated (d,l)-amphetamine-cation exchange resin complex and a modified release, barrier coated d-amphetamine-cation exchange resin complex are blended or admixed to provide the modified release component. In another embodiment, the modified release component contains a blend of barrier coated amphetamine-cation exchange resin complex-(optional matrix), in which a modified release, barrier coated d-amphetamine-cation resin complex and a modified release, barrier coated l-amphetamine-cation resin complex are blended or admixed to provide the modified release component. In yet another embodiment, the immediate release component includes an uncoated d-amphetamine-cation exchange resin complex and an uncoated l-amphetamine-cation exchange resin complex blended or admixed. In such an instance, the blend may comprise about 3.2% by weight d-amphetamine to about 1% by weight l-amphetamine, which weight percent is based on the weight of the amphetamine without calculating the weight contribution of the other components of the complex (e.g., ion exchange resin, or any matrix or coating polymer present). Following the complexation reaction, an amphetamine-cation exchange resin complex may be, in no particular order, milled to achieve a desired size range and dried (e.g., to a moisture content below about 10%, e.g., about 3% to about 7%), and then stored for future use. In one embodiment, the complex is milled or passed through a sieve to provide a particle size ranging from about 40 microns to about 4120 microns to enhance mouth feel (i.e., texture), or about 50 microns to about 250 microns. These particles may be either regularly or irregularly shaped. In some embodiments, the average particle size of the uncoated amphetamine-cation exchange resin complex or the average particle size of the coated amphetamine-cation exchange resin complex is milled to a size of about 100 to about 200 microns. These particle sizes may be determined using sieve analysis through a sieve shaker having USP standard wire mesh sieves conforming to ASTM specifications. Alternatively, other suitable methods known to those of skill in the art may be used to achieve this result.

In another embodiment, rather than drying for storage, a sufficient amount of water is removed from the wet mass containing the formed complex (i.e., the slurry in which the reaction occurred) to optimize uniform application of a granulating agent, followed by further removal of moisture. Typically, the washing steps, filtration, and/or other steps for removal of moisture result in removal of substantially all of counter-ions from the drug-cation exchange resin complex.

A number of the processing steps described herein, including, e.g., loading, pre-washing, complexing and granulation may be carried out in a multi-purpose apparatus, e.g., such as the AFD450 processor [Pall SeitzSchenk] or another similar and larger scale multi-purpose apparatus which is available commercially [e.g., by Rosenmund, U.S. Pat. No. 5,609,835]. The vessel is capable of or adapted for pivoting and has a single chamber with the capacity for handling a reaction/crystallization, filtration, resuspension, and drying. Such a vessel is typically provided with a water jacket connected with a thermostatically controlled heating and cooling system. Alternatively, this vessel is used for only a single processing step in conjunction with multiple different apparatus, including, e.g., granulators (e.g., the GMX-75 high shear granulator), a dryer, and others which are known in the art.

In one embodiment, a matrix forming polymer is combined with the amphetamine-cation exchange resin complex following only partial complexation, or by reducing the moisture content of the wet amphetamine-cation exchange resin complex to a range of between about 15% w/w to about 25% w/w, or another suitable amount. Treatment of the amphetamine-cation exchange resin complex with the matrix forming polymer is as follows.

### Amphetamine-Cation Exchange Resin Complex-Matrix

Optionally, a matrix-forming polymer is used to assist in processing an uncoated or precoated amphetamine-cation exchange resin complex. For example, a matrix-forming polymer may be used to facilitate granulation of the immediate release amphetamine-cation exchange resin complex, or as desired, e.g., in preparation for coating of an uncoated amphetamine-cation exchange resin complex.

In one embodiment, a polyvinylpyrrolidone polymer [e.g., such as may be purchased commercially as KOLLIDON® 30] is combined with the amphetamine-cation exchange resin complex in order to facilitate granulation prior to coating. Other hydrophilic polymeric granulating agents may include water-soluble polymeric materials which have been described in the art as impregnating agents or solvating agents and which function in the present application as granulating agents. In one embodiment, the granulating agent is a polyethylene glycol. Examples of desirable impregnating/solvating agents include those described in US Published Patent Application 2007/0215511, Sep. 20, 2007, and US 2003/0099711, which are incorporated herein by reference, or in U.S. Pat. No. 4,221,778 and published US Patent application Publication No. US 2003/0099711, the disclosures of which are incorporated herein by reference. Specific examples of other impregnating agents include propylene glycol, polyethylene glycol, polyvinyl alcohol, hydroxypropyl methylcellulose, hydroxypropyl cellulose, and sorbitol.

Optionally, the amphetamine-cation exchange resin complexes of the present invention may be admixed or granulated with a water-soluble or water-insoluble polymer or a combination of a water-insoluble polymers prior to the application of the water-permeable diffusion barrier coating described herein. Upon admixture, these polymers do not form a separate layer on the amphetamine-cation exchange resin complex. These polymers form a matrix therewith. Examples of suitable matrix forming polymers include, for example, a polyvinyl acetate polymer or a mixture of polymers containing same (e.g., KOLLICOAT® SR 30D), cellulose acetates, ethylcellulose polymers (e.g., AQUACOAT™ ECD-30 or SURELEASE™), acrylic based polymers or copolymers (e.g., represented by the EUDRAGIT® polymer family of acrylic resins), cellulose phthalate, or any combination of such water-insoluble polymers or polymer systems. These matrix-forming polymers when used may further prolong or alter the release of the amphetamine from the cation exchange resin complex/matrix and maximize attaining the desired release profile. One suitable polymer is a polyvinyl acetate polymer as described herein or an acrylic polymer from the EUDRAGIT® family. Examples of suitable acrylic polymers from the EUDRAGIT® family may include, e.g., a copolymer comprising ethyl acrylate and methyl methacrylate (e.g., EUDRAGIT® NE-30D), or EUDRAGIT® RS30D, RL30D, which are largely pH-independent polymers. EUDRAGIT® RS30D is a 30% aqueous dispersion of poly (ethyl acrylate-co-methyl methacrylate-co-trimethylammonioethyl methacrylate chloride) in a ratio of 1:2:0.1; other aqueous dispersions of this copolymer may be selected. Eudragit® RL30 D is a 30% aqueous dispersion of poly (ethyl acrylate-co-methyl methacrylate-co-trimethylammonioethyl methacrylate chloride) 1:2:0.2; other aqueous dispersions of this copolymer may be selected for use in the invention. Although less desirable, certain pH-dependent (enteric) polymers including, e.g., members of the EUDRAGIT polymer family, e.g., the L, S, and E, polymers and others which are commercially available may be selected.

The quantity of matrix forming polymer in an amphetamine-cation exchange resin complex-matrix typically ranges from about 1% to about 30%, or about 3 to about 20%, or about 3 to about 10%, about 10% to about 15%, about 15 to 25%, or about 5% or more by weight of the uncoated or amphetamine-cation exchange resin particulates prior to their being optionally coated. However, higher or lower amounts may be selected. In one embodiment, where it is desired for the matrix forming polymer to have little or no effect on release rate, a hydrophilic polymer may be selected and used in a higher amount, whereas a hydrophobic release retardant if selected for use will be used at a lower amount. Following admixing, the amphetamine-cation exchange resin complex particles with the matrix forming polymer, the mixture is dried and the amphetamine-cation exchange resin complex-matrix granules are milled appropriately to the desired particulate size.

For the amphetamine-cation exchange resin complex-matrix which will be coated and the uncoated amphetamine-cation exchange resin complex, the particles are milled though a size below about 420 microns, or generally in the range of about 50 microns to about 420 microns, or about 100 microns to about 420 microns. This can be achieved, e.g., using a CO-MIL device fitted with a 40 mesh screen. In one embodiment, the particles have an average size of about 100 to about 250 microns, or about 100 to about 200 microns. In some cases, the milling may be carried out before the complete drying of the complex or complex matrix and then again further drying followed by milling to obtain the desired complex characteristics. These particle sizes maybe determined using sieve analysis through a sieve shaker having USP standard wire mesh sieves conforming to ASTM specifications.

### Barrier Coat for Modified Release

The modified release component of an amphetamine-cation exchange resin complex-matrix with a barrier coating which modifies the release profile of the active amphetamine(s) such that the amphetamine has at least about a modified release profile. Suitably, the barrier coating has a pH-independent release (i.e., it is not an enteric coating which has pH-dependent release) and is a water-insoluble, water-permeable coating material. In a preferred embodiment, the chewable tablet lacks an enteric or other pH-dependent coating.

Suitably the properties of the barrier coating provide modified release properties to the barrier coated amphetamine-cation exchange resin complex, which is amphetamine-cation exchange resin is optionally in matrix. When the matrix is present, the barrier coating is applied over the amphetamine-cation exchange resin complex-matrix. The barrier coating provides the modified release component with resistance to grinding forces which enables the portions of a chewable ER tablet to provide a modified release amphetamine profile even when cut into pieces.

In one embodiment, the barrier coating has a characteristic high flexibility or elongation (elasticity) at break measured by the texture analyzer TA-XT2 HiR (Stable Microsystems) and by the method reported by the manufacturer in its literature [i.e., Jan-Peter Mittwollen, Evaluation of the Mechanical Behavior of Different Sustained Release Polymers, Business Briefing: Pharmagenerics, 2003, pp. 1-3, BASF]. In certain embodiments, the modified release barrier coating layer comprises a modified release polymeric coating material, such as described in more detail below, and at least one plasticizer incorporated into the barrier coating polymer to enhance high flexibility or elongation (elasticity) of the polymeric film coating. Elongation at break may be measured by the texture analyzer TA-XT2 HiR (Stable Microsystems) and/or by the method reported by the manufacturer in its literature [i.e., Jan-Peter Mittwollen, Evaluation of the Mechanical Behavior of Different Sustained Release Polymers, Business Briefing: Pharmagenerics, 2003, pp. 1-3, BASF] and maybe at least about 100%, of at least about 125% and preferably in a range between about 150% to about 400% as compared to the original polymeric film. Suitably, the plasticizer is selected so as to not substantially increase the tackiness of the polymer film greater than about 2 (wherein the film is measured by the Hossel method referenced above independent of any composition on which it has been deposited). Generally, a plasticizer is used in the percent range, or a mixture of plasticizers combine to total about 2 to about 50% by weight of the coating layer, more preferably about 2.5% to about 30% by weight, or about 5% to about 20% by weight of the coating layer on the coated amphetamine-cation exchange resin complex.

In one embodiment, the barrier coating layer is about 10% to about 55%, by weight, or about 10% to about 50%, by weight, of the amphetamine-cation exchange resin complex-optional matrix in order to provide the modified release component. In another embodiment, the barrier coating layer is about 20% to about 40%, about 25% to about 35% by weight, or about 30%, by weight of the amphetamine-cation exchange resin complex-matrix (i.e., prior to coating). Still other suitable ranges can be determined by one of skill in the art, having been provided with the information herein.

The barrier coating is applied over the uncoated amphetamine-cation exchange complex-optional matrix (e.g., as an aqueous dispersion or a solution), dried, and milled or passed through a screen such that the barrier coated amphetamine-cation exchange complex-optional matrix particles are in the same size range as described in the preceding paragraph, i.e. an average of about 100 microns to about 300 microns, or an average of about 150 microns to about 250 microns.

In one embodiment, the barrier coating is applied as an aqueous dispersion which is dried in order to provide the desired modified release profile. In the case of an aqueous-based polyvinylacetate coating, the coating is cured in order to provide the desired release profile.

In one embodiment, the barrier coating is applied as an aqueous dispersion of a water insoluble polymer comprising a polyvinyl acetate polymer, or a blend of polymers comprising a polyvinyl acetate polymer. In one embodiment, the barrier coating further contains a plasticizer, which can facilitate uniform coating of the amphetamine-cation exchange resin complex-optional matrix and enhances the tensile strength of the barrier coating layer.

One coating composition useful in the amphetamine compositions is applied in the form of an aqueous dispersion containing polyvinylacetate (PVA) polymer based aqueous coating dispersion and a plasticizer. The PVA is insoluble in water at room temperature. The PVA may be used in either substantially pure form or as a blend. Where the barrier coating comprises a PVA polymer, the PVA polymer is present in an amount of about 70% to about 90% w/w of the final barrier coating layer, at least about 75%, at least about 80%, about 85% w/w of the final barrier coating layer. Generally, a plasticizer is used in the percent range, or a mixture of plasticizers combine to total about 2 to about 50% by weight of the coating layer, more preferably about 2.5% to about 20% by weight of the coating layer on the coated amphetamine-cation exchange resin complex. Preferably a plasticizer is in a range of about 2.5 to about 15% by weight of the coating layer based on the coated complex provides the most desirable properties. Suitable plasticizers may be water soluble and water insoluble. Examples of suitable plasticizers include, e.g., dibutyl sebacate, propylene glycol, polyethylene glycol, polyvinyl alcohol, triethyl citrate, acetyl triethyl citrate, acetyl tributyl citrate, tributyl citrate, triacetin, and Soluphor® P (2-pyrrolidone), and mixtures thereof. Other plasticizers are described in patent application publication US 2003/0099711 A1, May 29, 2003, page 4 (0041) the disclosure of which is incorporated herein by reference.

A commercial polyvinylacetate blend contains primarily a polyvinylacetate polymer, a stabilizer, and minor amounts of a surfactant such as sodium lauryl sulfate. Where the barrier coating comprises PVP as the stabilizer component, the final barrier coating layer generally contains about 5 to about 10% w/w of polyvinyl pyrrolidone. In one desired embodiment, the aqueous based barrier coating solution is KOLLICOAT® SR 30 D (BASF Corporation) and whose composition is about 27% PVA polymer, about 2.7% polyvinylpyrrolidone (PVP), about 0.3% sodium lauryl sulfate (solids content 30% w/w), mixed with a plasticizer. See, also, U.S. Pat. Nos. 6,066,334 and 6,026,277, which is incorporated by reference herein. The PVP and surfactant help stabilize the aqueous dispersion of the PVA. Generally, such stabilizing components are present in an amount totaling less than about 10% w/w, and preferably less than about 5% w/w. Optionally, a selected surfactant is present in an amount of about 1% or less. In one embodiment, the surfactant is a non-ionic surfactant. Optionally, an ionic surfactant may be selected.

In a particularly desirable embodiment, the desired modified release is obtained when the coating layer formed by application of the aqueous dispersion containing the KOLLICOAT® SR-30D-plasticizer is dried and cured. Preferably, the coating is cured for about 1 to about 24 hours. In alternate embodiments, the coating is cured for about 4 to about 16 hours, and preferably about 5 hours at high temperature, e.g., about 50° C. to about 65° C., and preferably about 60° C. Thus, in one embodiment, the coated amphetamine-cation exchange resin complex-matrix has a cured water-permeable, high tensile strength, water insoluble, barrier coating comprising a non-ionic polymer and a plasticizer and having an elongation factor in the range of about 150% to 400% over the amphetamine-cation exchange resin complex-matrix. In one embodiment, the barrier coating comprises a polyvinylacetate polymer, a stabilizer, a surfactant and a plasticizer. In one embodiment, a barrier coating comprises about 2.5 to about 30% of plasticizer, about 70 to about 90% polyvinylacetate, about 5 to about 10% polyvinylpyrrolidone, and about 0.1 to about 1% surfactant. See, e.g., US Published Patent Application No. US 2007/0215511A, published Sep. 20, 2007, and its counterpart application, WO 2007/109104, the disclosures of which are incorporated herein by reference.

It may be possible utilize other aqueous or non-aqueous solvent based systems which do not require curing. For example, an aqueous based acrylic polymer (a EUDRAGIT® RL30D and EUDRAGIT® RS30D blend is described herein), but requires the addition of anti-tacking agent such as, e.g., talc or glycerol monostearate (GMS), in order to facilitate processing and even coating.

In one embodiment, the coating may be a EUDRAGIT® brand acrylate based coating materials [including, e.g., a poly (ethyl acrylate-co-methyl methacrylate-co-trimethylammonioethyl methacrylate chloride) polymer system]. For example, EUDRAGIT® RS 30D [a pH-independent, 30% aqueous dispersion of poly (ethyl acrylate-co-methyl methacrylate-co-trimethylammonioethyl methacrylate chloride) 1:2:0.1)], or EUDRAGIT® RL 30D [a 30% aqueous dispersion, pH independent polymer, poly (ethyl acrylate-co-methyl methacrylate-co-trimethylammonioethyl methacrylate chloride) 1:2:0.2)] may be selected as the barrier coating. In one embodiment, a blend of EUDRAGIT® RS 30D and EUDRAGIT® RL 30D may be prepared to optimize the hydrophilicity/hydrophobicity of the film in order to achieve desirable release profiles.

Suitably, a plasticizer may be included in the coating layer. In one embodiment, the barrier coating comprises about 2 to about 50% by weight of the coating layer, more preferably about 2.5% to about 2030% by weight, or about 5% to about 20% by weight of the barrier coating layer. Individual or a combination of hydrophilic or lipophilic plasticizers with a dispersion or suspension containing the barrier coating polymer. Such plasticizers include, e.g., propylene glycol, polyethylene glycol, triacetin, triethyl citrate, dibutyl sebacate, vegetable oil, lipids, etc. Optionally, a suitable anti-tacking agent may be mixed with one of the EUDRAGIT® products to improve flow during coating and to address issues of tackiness of the product during processing. Suitable anti-tacking agents include, e.g., talc, glycerol monostearate (GMS), and mixtures thereof. Suitably, these agents are present in an amount of about 0.2%-4.5% w/w based on the dry weight of the coating polymer applied to form the coating layer of the modified release component. Typically, the coating layer resulting from application of the blend described in this paragraph is not subject to any curing.

Optionally, another barrier coating may be selected. In another embodiment, a non-aqueous solvent-based ethylcellulose, such as commercially available as the ETHOCEL™ products (Dow) may be modified in order to achieve the barrier coating characteristics defined herein, e.g., by addition of a sufficient amount of plasticizer to improve flexibility and/or by curing to a sufficient temperature to achieve the desired release rate. Dow's web site describes three of these products, Std 7 (viscosity of 6-8 mPa-s (CP); Std 10 (9-11 mPa-s (CP)); Std 20 (18-22 mPa-S), each of which has a 48.0-49.5% ethoxyl content) as being useful for tablet coating. Further, optionally combining one of these polymers in combination with a water-soluble active and/or water-soluble excipient such as a METHOCEL™ cellulose ether and/or CARBOWAX™ polyethylene glycols is further described. Alternatively, it may be possible to modify an aqueous based ethylcellulose barrier coating in order to achieve the modified release barrier coating characteristics required herein, e.g., by addition of a sufficient amount of plasticizer to improve flexibility and/or by curing to a sufficient temperature to achieve the desired release rate. See, e.g., the barrier coatings described in U.S. Pat. Nos. 6,066,334 and 6,046,277; see, also, e.g., U.S. Pat. Nos. 6,046,277 and 6,001,392; US Published Patent Application No. 2003/0099711 and related application WO 03/020242; WO 2006/022996 and related applications US Published Patent Application Nos. US 2005/0232986; US 2005/0232987; US 2005/0232993; US 2005/0266032; U.S. Pat. Nos. 7,067,116; 6,667,058, 6,001,392, the disclosures of which are incorporated herein by reference, among others.

In certain embodiments, the water-permeable diffusion barrier is selected from the group consisting of ethyl cellulose, methyl cellulose and mixtures thereof. In certain embodiments, the coating material is SURELEASE®, manufactured by Colorcon which is water based ethyl cellulose latex, plasticized with dibutyl sebacate or with vegetable oils. Other non-limiting coating materials included within the scope of the present invention are AQUACOAT®, manufactured by FMC Corporation of Philadelphia, which is ethylcellulose pseudolatex; solvent based ethylcellulose; shellac; zein; rosin esters; cellulose acetate; EUDRAGIT® products, manufactured by Rohm and Haas of Philadelphia, which are acrylic resins; silicone elastomers; poly(vinyl chloride) methyl cellulose; and hydroxypropylmethyl cellulose.

A coating as described herein may be applied using techniques described by the polymer manufacturer and/or techniques which are known to those of skill in the art. Conventional coating solvents and coating procedures (such as fluid bed coating and spray coating) can be employed to coat the particles. Techniques of fluid bed coating are taught, for example, in U.S. Pat. Nos. 3,089,824; 3,117,027; and 3,253,944. The coating is normally applied to the drug/resin complex, but alternatively can be applied to the resin before complexing with the drug. See, e.g., U.S. Pat. Nos. 6,001,392; 3,089,824; 3,117,027; and 3,253,944. Non-limiting examples of coating solvents include ethanol, a methylene chloride/acetone mixture, coating emulsions, methyl acetone, tetrahydrofuran, carbon tetrachloride, methyl ethyl ketone, ethylene dichloride, trichloroethylene, hexane, methyl alcohol, isopropyl alcohol, methyl isobutyl ketone, toluene, 2-nitropropane, xylene, isobutyl alcohol, n-butyl acetate.

The coated amphetamine-cation exchange resin complex-(optional matrix) is then dried and/or cured. The dried, optionally cured, coated amphetamine-cation exchange resin complex-optional matrix may be passed through a suitable screen in order to ensure that the particle size is in the desired range, e.g., capable of passing through a standard 40 mesh screen. In one embodiment, the dried, optionally cured, coated amphetamine cation exchange resin complex (optional matrix) granules have a mean particle size in the range of about 100 microns to about 420 microns, or about 75 microns to about 250 microns, about 150 to about 300 microns. In one embodiment, less than 10% of the coated particles are under 75 microns in size.

### Finished Dose Formulations

In certain embodiments, the methods provided herein utilize amphetamine extended release chewable tablets as described herein. These compositions are orally administrable and readily titratable. The tablets maintain their extended release profile even when subject to chewing or division (e.g., splitting). In certain other embodiments, the methods provided herein utilize a bioequivalent amphetamine solid and/or liquid amphetamine extended release formulation. Preferably, the bioequivalent composition is bioequivalent through various intermediate timepoints between 0 hours to 10 hours, or up to 13 hours.

The one or more modified release barrier coated amphetamine-cation exchange resin complex-optional matrix, one or more uncoated amphetamine-cation resin complex and the one or more uncomplexed amphetamine may be prepared as described herein and blended together prior to combination with the excipients for formulation into a final chewable tablet formulation. Optionally, one or more of these components are prepared and dried (e.g., in powder form), either separately or together, for storage prior to formulation. Alternatively, the one or more modified release barrier coated amphetamine-cation exchange resin complex-optional matrix, one or more uncoated amphetamine-cation resin complex and the one or more uncomplexed amphetamine may be prepared separately as described above, and combined in any suitable order with the excipients present in the aqueous suspension base and/or the chewable tablet.

In one aspect the methods utilize an amphetamine extended release chewable tablet, which is optionally also orally dissolving. Following administration of a single dose of the oral amphetamine extended release chewable tablet, in some embodiments, a therapeutically effective amount of amphetamine is reached in less than about thirty minutes, and as soon as about twenty, ten, or fewer minutes, and the formulation provides an extended release therapeutically effective profile to at least about 12 hours, or about 13 hours, or up to about 18 hours. Further, the chewable tablet may be scored, as dividing the tablet does not significantly modify the in vitro profile of the tablet portions resulting from split or other division of the intact tablet. Further, because the amphetamine chewable tablet described herein retains its extended release properties even when scored, it can be divided into other suitable portions. This makes the chewable tablet readily titratable and convenient for physicians to reduce the dose for patients to introduce the drug in a smaller dose or incremental doses of medication for patients whose needs dictate such. This ability to divide the dose into portions allows physicians to take into consideration individual patient needs, including factors like age, body weight and individual response to the medication without the need for taking multiple doses over a twelve hour period of another product which offers only immediate release.

The amphetamine extended release chewable tablets are typically dry blended prior to being compressed into a chewable tablet. Suitably, the chewable tablet is solid blend which provides extended release properties even when scored such that when divided the separated tablet portions retain the extended release profile described herein. In certain embodiments, the tablets have a uniformly blended core containing all the active amphetamine components and are distinguished from tablets layered with different actives.

In one embodiment, the chewable tablet has a hardness of about 2.5 kilopond (kp) to about 20 kp or about 3 to about 15 kp. One (1) kilopond is one kilogram of force (kgf). Newtowns (N) are the SI unit of force and the SI standard for tablet hardness testing. 1 kilopond (kp) is equal to 9.80665 Newtons (N). Presented in Newton rounded to the nearest five, the chewable tablet has a hardness of about 45 N to about 245 N, about 75 N to about 200 N, or about 95 N to about 160 N. Optionally, the hardness may be dose proportional, with lower doses having lower hardness levels. For example, a 20 mg chewable tablet may have a hardness in the range of about 7 to about 14 kp (about 68 N to about 127 N), a 15 mg tablet may have a hardness in the range of about 3 to about 9 kp (about 29 N to about 88 N), and a 10 mg tablet may have a hardness in the range of about 3 kp to about 11 kp (about 29 N to about 108 N). In one embodiment, the hardness is determined following compression and prior to application of any color or other non-functional tablet coating as defined herein. In one embodiment, the tablet portions meet the USP Friability requirement. In one embodiment, the friability of both the intact tablet and the tablet portions are less than about 1.

In certain embodiments, a composition useful in the methods provided herein are distinguished by being chewable and also being a rapid dissolve tablet or an orally dissolving tablet (ODT) that will dissolve in the mouth in less than 1 minute. An ODT has been defined as a solid dosage form containing medicinal substances that disintegrates rapidly, usually within a matter of seconds, when placed upon the tongue. In general, the total weight of the ODT is recommended not to exceed 500 mg. See, Guidance for Industry, Orally Disintegrating Tablets, US Department of Health and Human Services, Food and Drug Administration Center for Drug Evaluation and Research (CDER) December 2008. In one embodiment, the tablet provided herein is a unit dosage form which is about 375 mg to about 425 mg, or about 400 mg in total weight. In another embodiment, the tablet is a unit dosage form which is about 350 mg to about 375 mg, or about 360 mg in total weight. In a further embodiment, the tablet is a unit dosage form which is about 200 to about 250 mg, or about 240 mg in total weight.

In certain embodiments, the tablet may be an orally disintegrating tablet. In certain embodiments, the tablet may be an orally dispersible tablet.

In one embodiment, the excipients do not alter the modified release properties of the chewable tablet. The modified release profile is provided by the modified release barrier coated amphetamine-cation exchange resin complex-matrix component. The amphetamine components may be pre-blended in the desired ratio to one another prior to being admixed with the excipients, described below. Alternatively, each of the amphetamine components are added separately and blended with the excipients.

The amphetamine extended release chewable tablets may be prepared using one or more of a filler, one or more disintegrant, one or more binder, one or more a buffering agent, one or more lubricant, one or more glidant, or blends of these components. Suitably, the tablets also include taste and/or mouth feel enhancers including, e.g., one or more of a sweetener, a flavorant, a gum, or blends of these components. Optionally, the tablet may also contain a non-functional coating.

As used herein, a “non-functional coating” refers to a coating which contributes no detectable modified release functions. The non-functional coating may be a polymer which may serve as a moisture barrier to preserve the integrity of the tablet during storage or to facilitate application of a color coating layer. Additionally or alternatively, the non-functional coating may provide a color coating layer or improve the “smoothness” or mouth feel of the tablet. In one embodiment, the non-functional coating may increase the hardness of the tablet somewhat without affecting the chewability thereof.

Throughout the specification, where weight percentages of excipients and the active components are provided, the weight percentages are exclusive of any weight added by a non-functional coating. Weight percentages of these non-functional coatings, where present, are provided as weight added, in an amount of about 1% to about 20%, or about 2% to about 10%, or about 3% to about 5% weight added to the finished chewable tablet.

A chewable tablet may contain a filler or a mixture of fillers in the range of about 50% w/w to about 90% w/w, about 55% w/w to about 85% w/w, or about 60% w/w to about 70% w/w of the total tablet weight, or values therebetween. Suitable fillers may include, e.g., mannitol, lactose, maltose, fructose, sucrose, xylitol, maltitol, microcrystalline cellulose, dicalcium phosphate, guar gum, xanthan gum, tragacanth gum, pre-gelatinized starch, compressible sugar, calcium carbonate, magnesium carbonate, calcium sulfate, dextrates, maltodextrin. In one embodiment, a chewable tablet contains a blend of mannitol, xanthan gum, microcrystalline cellulose, and guar gum in an amount of about 60% w/w to about 75% w/w. In one embodiment, a gum or a combination of gums is provided in an amount of about 0.25% w/w to about 5% w/w, or about 0.25% to about 1% w/w. In another embodiment, microcrystalline cellulose is provided in an amount of about 10% w/w to about 25% w/w, or about 15% w/w to about 20% w/w based on the total tablet weight prior to any non-functional coating. A product containing a combination of microcrystalline cellulose and guar gum is commercially available as Avicel®, which contains a ratio of 80 parts by weight microcrystalline cellulose to 20 parts by weight guar gum. This blend of microcrystalline cellulose (MCC) and guar gum may be present in an amount of about 10% w/w to about 20% w/w of the total tablet weight.

A chewable tablet as described herein will also contain a disintegrant or blend of disintegrants in the range of about 1% w/w to about 25% w/w, or about 5% w/w to about 15% w/w, or about 10% w/w to about 14% w/w based on the total tablet weight. Suitable disintegrants include, e.g., crospovidone, sodium starch glycolate, croscarmellose sodium, carboxymethyl cellulose sodium, carboxymethylcellulose calcium, starch. In one embodiment, a tablet as described herein contains crospovidone in a range of about 5% w/w to about 10% w/w, or about 12% w/w based on the tablet weight prior to any non-functional coating being applied.

The binder for the chewable tablet may be absent (i.e., 0%), or optionally, present in an amount of about 1% w/w to about 15% w/w of the total tablet weight. Examples of suitable binders include polyvinylpyrrolidone (Povidone), hydroxypropyl methyl cellulose, hydroxypropyl cellulose, hydroxyethyl cellulose, methyl cellulose, polyvinyl alcohol, starch, acacia, alginic acid, sodium alginate.

In one embodiment, the chewable tablet contains a sweetener in an amount of about 0.010% w/w to about 30% w/w, or about 0.50% w/w to about 2% w/w, or about 1% w/w to about 2% w/w or about 1.5% w/w, based on the total tablet weight exclusive of any optional non-functional coating. Suitable sweeteners may include, e.g., Aspartame, Saccharin, Saccharin sodium, Sucralose, Sodium cyclamate, Xylitol, Acesulfame Potassium, and blends thereof. Optionally, in addition to functioning as a sweetener, an excipient may function as a filler. Examples of suitable sweeteners/fillers including, e.g., fructose, sucrose, xylitol, maltitol. Optionally, when performing both functions, the excipient may be present in an amount in excess of about 10% w/w of the tablet. In such an instance, additional sweetener may be omitted (e.g., present in 0% added sweetener). Alternatively, a second sweetener or a combination of sweeteners which differs from the filler is added in the amount provided in this paragraph in order to further enhance taste.

Suitably, the tablet may contain a buffering agent in an amount of about 0.1% w/w to about 5% w/w, or about 0.5% w/w to about 1.5% w/w based on the total tablet weight. Examples of suitable buffering agents include, e.g., citric acid, tartaric acid, malic acid, lactic acid, and acceptable salts thereof, and mixtures thereof. In one embodiment, the buffering agent adjust the pH of the tablet (if suspended in water) to a range of about 5 to about 6.

When an additional flavoring agent is added, the flavoring agent(s) may be added in an amount of about 0.05% w/w to about 3% w/w, or about 0.1% to about 1% w/w or about 0.5% w/w, based on the total weight of the tablet (exclusive of any optional non-functional coating). Suitable flavoring agents may include, both natural and artificial flavoring agents such as are generally available through several custom manufacturers around the world such as Fona [Illinois, US], Givaudan (Vernier, Switzerland), Ungerer & Company (Lincoln Park, N.J.), and International Flavors & Fragrances (New York, N.Y.) to name a few. Those skilled in the art will recognize that there are several commercial sources available including custom blenders. The flavorings may be blended prior to addition to the pharmaceutical composition or added separately. Still other flavoring agents such as bubble gum, cherry, strawberry, vanilla, grape, banana and other flavors or mixtures thereof may be selected.

Optionally, a colorant may be provided to the tablet to provide a desired visual appeal or trade dress. Such colorants may be added in the range of about 0.001 to about 1% w/w, or about 0.01% w/w to about 0.08% w/w or about 0.05% w/w, based on the total weight of the tablet (exclusive of any non-functional coating). Such colorants are available from a variety of sources including, e.g., Colorcon, Noveon, and Spectra. In one embodiment, no colorants are used in the tablet.

In order to facilitate production of the chewable tablet, excipients such as lubricants and glidants may be utilized. A lubricant may be utilized in an amount of about 0.1% w/w to about 50% w/w, about 0.2% w/w to about 4.5% w/w, or about 1.50% w/w to about 30% w/w of the total weight of the tablet. Examples of lubricants may include, e.g., magnesium stearate, sodium stearyl fumarate, stearic acid, zinc stearate, calcium stearate, magnesium trisilicate, polyethylene glycol, and blends thereof. In one embodiment, A glidant may be used in an amount of about 0.01% w/w to about 0.5% w/w, or about 0.1% w/w to about 0.3% w/w, based on the total weight of the tablet. Examples of suitable glidants include, e.g., silicon dioxide and tribasic calcium phosphate. In one embodiment, the glidant is silicon dioxide which is used in an amount of about 0.001% w/w to about 0.3% w/w or about 0.2% w/w.

Optionally, other excipients may be selected from conventional pharmaceutically acceptable carriers or excipients and well established techniques. Without being limited thereto, such conventional carriers or excipients include diluents, binders and adhesives (i.e., cellulose derivatives and acrylic derivatives), lubricants (i.e., magnesium or calcium stearate, or vegetable oils, polyethylene glycols, talc, sodium lauryl sulfate, polyoxy ethylene monostearate), thickeners, solubilizers, humectants, disintegrants, colorants, flavorings, stabilizing agents, sweeteners, and miscellaneous materials such as buffers and adsorbents in order to prepare a particular pharmaceutical composition. The stabilizing agents may include preservatives and anti-oxidants, amongst other components which will be readily apparent to one of ordinary skill in the art.

The following Tables provides exemplary formulations of amphetamine extended release compositions according to the invention, based on the total weight of the tablet. In certain embodiments, 5 mg, 10 mg, 15 mg, and 20 mg tablet strengths are provided as dose weight proportional formulations. The tablets are designed to disintegrate after chewing followed by ingestion or after swallowing the whole tablet.

In certain embodiments, a single powder blend is used to prepare tablets of all four strengths. The powder blend contains two active ion exchange resin complexes: Coated (d, dl) AMP Polistirex and Uncoated (d,dl) AMP Polistirex, blended with a racemic Amphetamine salt, or a hydrate thereof, and a (d) Dextroamphetamine salt. These may be blended together further blending with the remaining excipients before compression into the final tablet.

In certain embodiments, the excipients are present in an amount as follows:

| | Quantity (mg/tablet) | | | | Quantity |
|---|---|---|---|---|---|
| Ingredients | 5 mg | 10 mg | 15 mg | 20 mg | % (w/w) |
| Amphetamine base | 5 | 10 | 15 | 20 | 1-5% |
| Complexing Agent (Cation | 10-40 | 20-80 | 35-120 | 60-150 | 5-20% |
| Exchange resin) | | | | | |
| Optional Matrix Forming | 0.33 | 0.66 | 0.99 | 1.3 | 0.01-0.5 |
| polymer/Binder | | | | | |
| Plasticizer | 0.05-0.1 | 0.1-0.4 | 10.2-0.5 | 10.2-0.6 | 0.001-0.1 |
| Modified Release Coating | 1-4 | 2-8 | 3-12 | 14-16 | 0.5-2% |
| Filler(s) | 70-276 | 138-552 | 210-830 | 276-1106 | 140-80 |
| Optional Sweetener | 1.5-6 | 3-12 | 4.5-18 | 6-20 | 1-3 |
| Optional Flavoring Agent | 0.5-2 | 1-4 | 1.5-6 | 2-8 | 0.025-1 |
| Disintegrant | 12-48 | 24-96 | 36-144 | 48-192 | 6-20 |
| Glidant(s) | 1.2-4.8 | 2.4-9.6 | 3.6-14.4 | 4.8-19.20 | 0.6-1.8 |
| Lubricant | 0.5-2 | 1-4 | 1.5-6 | 2-8 | 0.25-1 |

| | | |
|---|---|---|
| Component | Tablets % w/w | % w/w |

| | |
|---|---|
| Coated amphetamine polistirex | 4.583 |
| Uncoated amphetamine polistirex | 9.68 |
| Uncomplexed amphetamine | 2.947 |

In certain embodiments, the amphetamine polistirex contains racemic (dl)-amphetamine and (d)-amphetamine in weight ratio of about 0.75 to about 2, to about 1 to about 1.5. Further, in certain embodiments, the ratio of total amphetamines to cation exchange resin is about 1:1 to about 1:6, including ratios therebetween, such as 1:1.5, 1:2, 1:2.5, 1:3, 1:3.5, 1:4, 1:4.5, 1:5, 1:5.5, 1:6, or values therebetween. In other embodiments, other suitable loading amounts of amphetamine onto the resin may be selected.

In certain embodiments, the polistirex is prepared by complexing the (dl)-amphetamine and (d)-amphetamine and the cation exchange resin and granulating the amphetamine polistirex intermediate with a hydrophilic matrix-forming polymer prior to applying the coating. In certain embodiments, the coating is applied in an amount of about 15% w/w to about 40% coating weight gain, or about 20% w/w, or about 25% w/w, or about 30% w/w, or about 35% w/w, or about 40% w/w coating weight gain.

In certain embodiments, the tablets may be scored. For example, a 5 mg tablet may be scored to provide a 2.5 mg dose. However, regardless of scoring, the tablets may be chewed, dissolved, or swallowed whole by the patient.

Suitably, a chewable tablet useful in the methods provided herein is prepared as a single uniform solid blend. A typical manufacturing process for making a chewable tablet generally involves blending of the desired ingredients to form a uniform distribution of the coated amphetamine-cation exchange resin complex, the uncoated amphetamine-cation exchange complex, the uncomplexed amphetamine, and the excipients. If desired, a blend of the amphetamine components may be formed prior to blending into the excipient. The blend is then compressed into a single layer using standard methods and tablet presses such as well-known to those skilled in the art (e.g., Kilian, Fette, Korsch, Elizabeth, Sejong, Kikusui, SMI, Colton, Stok, and Manesty, amongst others.)

In one embodiment, a chewable tablet is produced in a round shape, caplet or capsule shape, optionally with a single bisect (a single scoring at the mid-line which facilitates splitting the tablet into halves). However, other shapes may be readily selected, including, e.g., a standard round shape, a flat faced shape, oval, bullet, square, triangle, diamond, pentagon, octagon, amongst others. Optionally, one or more of these tablet shapes may be provided with a quadrisect, i.e., two perpendicular scores which facilitate splitting the tablet into quarters.

Optionally, the chewable tablet may have one or more sealant or top coating which does not function to modify or extend the release of the amphetamine but which provides moisture barrier, of a color coating or other visual appeal. For example, such a coating may provide a “shine” to the tablet, enhance palatability, serve as an identifying color for the tablet, or other purposes. Such coatings are available commercially, e.g., from Colorcon or other suppliers. Typically, such a coating is composed of hydroxypropyl methylcellulose (HPMC) or polyvinylalcohol and is present in an amount of about 1% w/w to about 20% w/w, or about 2% w/w to about 10% w/w of the total tablet weight.

Suitably, the amphetamine extended release tablets can be scored without affecting the extended release profile. Thus, the oral dose is readily titrated, i.e., split in half, in order to readily and accurately deliver half the dose of the finished tablet. In one embodiment, a titratable, amphetamine chewable tablet of Example 2, and compositions bioequivalent thereto, are encompassed by the invention.

The finished compositions may be stored in glass or high density polyethylene (HDPE) bottles with or without a heat induced sealed (HIS) bottle. The bottle may also contain a desiccant. Alternatively, the tablets may be packaged into blister packs using standard methods well-known to those skilled in the art.

An in vitro dissolution test determines whether chewable tablets disintegrate within a prescribed time when placed in a dissolution media under prescribed experimental conditions. Disintegration is defined as the state in which no residue of the tablet, except fragments of undissolved coating, remains on the screen of the test apparatus or, if any other residue remains, it consists of a soft mass having no palpably firm, unmoistened core.

Examples of methods for in vitro testing of the amphetamine ER compositions have been described, e.g., by the World Health Organization (WHO) International Pharmacopoeia, (www.who.int/medicines/publications/pharmacopoeia/). An example of a suitable disintegration apparatus is described as follows. The apparatus consists of a circular basket-rack assembly, a suitable vessel for the immersion fluid (such as a 1-litre beaker), a thermostatic arrangement for maintaining the fluid at the required temperature (normally 37 2° C.), and a device for raising and lowering the basket-rack in the immersion fluid at a constant frequency of 28-32 cycles/min through a distance of 50-60 mm. The basket-rack assembly consists of six open-ended cylindrical glass tubes and a rack for holding them in a vertical position. The tubes are 75-80 mm long, and have an inside diameter of about 21.5 mm and a wall about 2 mm thick. The tubes are held vertically by two superimposed plates, circular in shape and made of transparent plastic material, each about 90 mm in diameter and 6 mm thick, perforated by six holes of a diameter that allows the tubes to be inserted. The holes are equidistant from the center of the plate and equally spaced one from another. A piece of woven gauze, made of stainless steel wire about 0.635 mm in diameter, with a mesh aperture of 2.0 mm is attached to the underside of the lower plate. The upper plastic plate is covered with a stainless steel plate, about 1 mm thick, of a diameter similar to that of the plastic plates. The steel plate is perforated by six holes about 22 mm in diameter, positioned to coincide with those of the upper plastic plate. It is placed over the tubes and consolidates the whole structure. The plates are held rigidly 75-80 mm apart by vertical stainless steel rods at the periphery. A metal rod is fixed to the center of the upper plate. This enables the assembly to be attached to a suitable mechanical device so that it may be lowered and raised. The volume of the fluid in the immersion vessel should be such that, at the highest point of the upward stroke, the wire mesh that forms the bottom of the basket remains at least 25 mm below the surface of the fluid. At the lowest point of the downward stroke, it should descend to not less than 25 mm from the bottom of the vessel. The time required for the upward stroke should be equal to the time required for the downward stroke, and the change in stroke direction should be a smooth transition rather than an abrupt reversal of motion. Where a disc is prescribed in the monograph, the following configuration and dimensions apply: a cylindrical disc 20.7±0.15 mm in diameter and 9.5±0.15 mm thick, made of transparent plastic with a relative density of 1.18 to 1.20. Each disc is pierced by five holes 2 mm in diameter, one in the center and the other four spaced equally on a circle of radius 6 mm from the center of the disc. On the lateral surface of the disc, four equally spaced grooves are cut so that on the upper surface of the disc they are 9.5 mm wide and 2.55 mm deep and, at the lower surface, 1.6 mm square. Different designs of basket-rack assembly may be used, provided that the specifications for the glass tubes and the stainless steel wire gauze are maintained. In vitro dissolution of the amphetamine compositions described herein may be assessed through a variety of methods including, e.g., Food and Drug Administration (FDA)-accepted dissolution tests, including the Basket Method (I) approved for use with an amphetamine chewable tablet, www_accessdata_fda_gov/scripts/cder/dissolution/-dsp_SearchResults_Dissolutions_cfm, have been described. The current FDA-approved dissolution test for a prior art amphetamine tablet comprising Amphetamine Aspartate/Amphetamine Sulfate/Dextroamphetamine Saccharate/Dextroamphetamine Sulfate utilizes water and a paddle speed of 100 rpm, at 500 mL, with testing at 10, 20, 30 and 45 minutes; another prior art tablet containing dextroamphetamine sulfate uses the same test and volume, with testing at 10, 20, 30, 45 and 60 minutes. Two extended release capsules involve testing with Basket Method (II), 50 rpm, in a media of dilute HCl (pH 1.1 for first 2 hrs, then add 200 mL of 200 mM Phosphate Buffer and adjust to pH 6.0 for the remainder) and volume of 750 mL for 0-2 hours and after 2 hours 950 mL; with testing at 1, 2, 3, 4, and 4 or 6 hours (amphetamine ER). Other amphetamine products have different dissolution tests and different media. However, since the barrier coated amphetamine-cation exchange complex described herein is not readily soluble in water, the dissolution medium for the previously described tests is not suitable for testing dissolution of the present tablets at those time frames. Accordingly, a dissolution medium of phosphate buffer described below may be used to assess in vitro dissolution of the amphetamine compositions described herein rather than water.

In one embodiment, an orally administrable extended release amphetamine composition provides for d-amphetamine, following a single dose of the composition as determined following a chewed dose comprising the equivalent to 20 mg total amphetamine: an arithmetic mean AUC_{0-∞} of about 965 to about 1508 ng-h/mL, a geometric mean AUC_{0-∞} of about 931 ng-h/mL to about 1455 ng-h/mL h/mL, an arithmetic mean AUC₀₋₂ of about 24.8 ng-h/mL to about 38.79 ng-h/mL or a geometric mean AUC₀₋₂ of about 22.85 ng-hr/mL to about 35.72 ng-hr/mL, an arithmetic mean AUC₀₋₅ of about 138 ng-h/mL to about 216 ng-h/mL or a geometric mean AUC₀₋₅ of about 134 ng-hr/mL to about 209 ng-hr/mL, an arithmetic mean AUC₅₋ₜ 767 ng-hr/mL to about 1199 ng-hr/mL or a geometric mean AUC₅₋ₜ of about of about 740 ng-h/mL to about 1156 ng-h/mL, an arithmetic mean Cₘₐₓ of about 44 ng/mL to about 69 ng/mL, a geometric mean Cₘₐₓ of about 43 ng/mL to about 68 ng/mL, or a Tₘₐₓ of about 3 hours to about 7 hours.

In one embodiment, an orally administrable extended release amphetamine composition provides one or more of the pharmacokinetic parameters for d-amphetamine, following a single dose of the composition is as determined for a whole swallowed tablet dose comprising the equivalent to 20 mg total amphetamine:an arithmetic AUC_{0-∞} of about 972 to about 1519 ng-h/mL, a geometric mean AUC_{0-∞} of about 948 ng-h/mL to about 1481 ng-h/mL h/mL, an arithmetic mean AUC₀₋₂ of about 26.30 ng-h/mL to about 41.10 ng-h/mL or a geometric mean AUC₀₋₂ of about 24.48 ng-hr/mL to about 38.25 ng-hr/mL, an arithmetic mean AUC₀₋₅ of about 141 ng-h/mL to about 220 ng-h/mL or a geometric mean AUC₀₋₅ of about 138 ng-hr/mL to about 215 ng-hr/mL, an arithmetic mean AUC₅₋ₜ of about 768 ng-h/mL to about 1200 ng-h/mL, or a geometric mean AUC₅₋ₜ of about 749 ng-hr/mL to about 1170 ng-hr/mL, an arithmetic mean Cₘₐₓ of about 42 ng/mL to 66 ng/mL, a geometric mean Cₘₐₓ of about 42 ng/mL to 66 ng/mL, an arithmetic mean or a Tₘₐₓ of about 2 hours to about 9 hours.

In one embodiment, an orally administrable extended release amphetamine composition provides, following a single dose of the tablet comprises one or more of the pharmacokinetic parameters for d-amphetamine following dosing an adult under fasted conditions with the tablet comprising the equivalent of 20 mg total amphetamines: for a chewed dose: an arithmetic mean AUC_{0-∞} of about 1206 ng-h/mL, a geometric mean AUC_{0-∞} of about 1164 ng-hr/mL, an arithmetic mean AUC₀₋₂ of about 31 ng-h/mL, a geometric mean AUC₀₋₂ of about 29 ng-hr/mL, an arithmetic mean AUC₀₋₅ of about 173 ng-h/mL, a geometric mean AUC₀₋₅ of about 167 ng-hr/mL, an arithmetic mean AUC₅₋ₜ of about 959 ng-h/mL, a geometric mean AUC₅₋ₜ of about 925 ng-hr/mL, an arithmetic mean Cₘₐₓ of about 55 ng/mL, a geometric mean Cₘₐₓ of about 54 ng/mL, or a Tₘₐₓ of about 5 hours.

In one embodiment, an orally administrable extended release amphetamine composition provides for d-amphetamine, following a single dose of the tablet comprises, for a whole swallowed dose: for a whole swallowed dose: an arithmetic mean AUC_{0-∞} of about 1215 ng-h/mL, a geometric mean AUC_{0-∞} of about 1185 ng-hr/mL, an arithmetic mean AUC₀₋₂ of about 33 ng-h/mL, a geometric mean AUC₀₋₂ of about 31 ng-hr/mL, an arithmetic mean AUC₀₋₅ of about 176 ng-h/mL, a geometric mean AUC₀₋₅ of about 172 ng-hr/mL, an arithmetic mean AUC₅₋ₜ of about 960 ng-h/mL, a geometric mean AUC₅₋ₜ of about 936 ng-hr/mL, an arithmetic mean Cₘₐₓ of about 53 ng/mL, a geometric mean Cₘₐₓ of about 53 ng/mL, or a Tₘₐₓ of about 5 hours.

In one embodiment, an orally administrable extended release amphetamine composition provides, following a single dose of the tablet, one or more of the pharmacokinetic parameters for l-amphetamine following dosing an adult under fasted conditions with the tablet comprising the equivalent of 20 mg total amphetamines: as determined following a chewed dose: as determined following a chewed dose: an arithmetic mean AUC_{0-∞} of about 378 to about 591 ng-h/mL, a geometric mean AUC_{0-∞} of about 362 ng-h/mL to about 566 ng-h/mL, an arithmetic mean AUC₀₋₂ of about 7.57 ng-h/mL to about 11.85 ng-h/mL or a geometric mean AUC₀₋₂ of about 6.97 ng-hr/mL to about 10.89 ng-hr/mL, an arithmetic mean AUC₀₋₅ of about 43 ng-h/mL to about 68 ng-h/mL, a geometric mean AUC₀₋₅ of about 42 ng-hr/mL to about 65 ng-hr/mL, an arithmetic mean AUC₅₋ₜ 292 ng-hr/mL to about 456 ng-hr/mL, a geometric mean AUC₅₋ₜ of about 280 ng-h/mL to about 438 ng-h/mL, an arithmetic mean Cₘₐₓ of about 13.6 ng/mL to about 21.25 ng/mL, a geometric mean Cₘₐₓ of about 3.6 ng/mL to about 21.25 ng/mL, or a Tₘₐₓ of about 3 hours to about 7 hours.

In one embodiment, an orally administrable extended release amphetamine composition provides, following a single dose of the tablet, one or more of the pharmacokinetic parameters for l-amphetamine following a whole swallowed dose: an arithmetic mean AUC_{0-∞} of about 385 ng-h/mL to about 601 ng-h/mL, a geometric mean AUC_{0-∞} of about 373 ng-h/mL to about 583 ng-h/mL h/mL, an arithmetic mean AUC₀₋₂ of about 8.09 ng-h/mL to about 12.64 ng-h/mL or a geometric mean AUC₀₋₂ of about 7.49 ng-hr/mL to about 11.71 ng-hr/mL, an arithmetic mean AUC₀₋₅ of about 44 ng-h/mL to about 69 ng-h/mL, a geometric mean AUC₀₋₅ of about 43 ng-hr/mL to about 68 ng-hr/mL, an arithmetic mean AUC₅₋ₜ of about 294 ng-hr/mL to about 459 ng-hr/mL, a geometric mean AUC₅₋ₜ of about 286 ng-h/mL to about 446 ng-h/mL, an arithmetic mean Cₘₐₓ of about 13.6 ng/mL to about 21.25 ng/mL, a geometric mean Cₘₐₓ of about 3.6 ng/mL to about 21.25 ng/mL, or a Tₘₐₓ of about 2 hours to about 9 hours.

In one embodiment, an orally administrable extended release amphetamine composition provides, following a single dose of the tablet, one or more of the pharmacokinetic parameters for l-amphetamine following dosing an adult under fasted conditions with the tablet comprising 20 mg total amphetamines: for a chewed dose: an arithmetic mean AUC_{0-∞} of about 473 ng-h/mL, a geometric mean AUC_{0-∞} of about 453 ng-hr/mL, an arithmetic mean AUC₀₋₅ of about 54 ng-h/mL, a geometric mean AUC₀₋₅ of about 52 ng-hr/mL, an arithmetic mean AUC₅₋ₜ of about 365 ng-h/mL, a geometric mean AUC₅₋ₜ of about 350 ng-hr/mL, an arithmetic mean Cₘₐₓ of about 17 ng/mL, a geometric mean Cₘₐₓ of about 17 ng/mL, or a median Tₘₐₓ of about 5 hours;

In one embodiment, an orally administrable extended release amphetamine composition provides, following a single dose of the composition, for a whole swallowed dose: an arithmetic mean AUC_{0-∞} of about 481 ng-h/mL, a geometric mean AUC_{0-∞} of about 466 ng-hr/mL, an arithmetic mean AUC₀₋₅ of about 55 ng-h/mL, a geometric mean AUC₀₋₅ of about 54 ng-hr/mL, an arithmetic mean AUC₅₋ₜ of about 367 ng-h/mL, a geometric mean AUC₅₋ₜ of about 357 ng-hr/mL, an arithmetic mean Cₘₐₓ of about 17 ng/mL, a geometric mean Cₘₐₓ of about 17 ng/mL, or a median Tₘₐₓ of about 5 hours.

In certain embodiments, the extended release amphetamine solid composition provides a plasma pharmacokinetic profile for d-amphetamine which is about 80% to about 125% of one or more of one or more: a geometric mean AUCₜ of about 821 ng-h/mL, a geometric mean AUC_{inf} of about 1013 ng-h/mL, a geometric mean Cₘₐₓ of about 53 ng/mL, a geometric mean Tₘₐₓ of about 3.85, an arithmetic mean AUCₜ of about 857 ng-h/mL, an arithmetic mean about AUC_{inf} of about 1061 ng-h/mL, an arithmetic mean Cₘₐₓ of about 55 ng/mL, or an arithmetic mean Tₘₐₓ of about 4, as measured in children 6-12 years old under fasting conditions following dosing with the composition having the equivalent of 20 mg of amphetamine base; an arithmetic mean AUC₀₋₄ of about 144 ng-h/mL, an arithmetic mean AUC₄₋ₜ of about 1000 ng-h/mL, an arithmetic mean AUC₀₋₅ of about 196 ng-h/mL, an arithmetic mean AUC₅₋ₜ of about 948 ng-h/mL, a geometric mean AUC₀₋₄ of about 139 ng-h/mL, a geometric mean AUC₄₋ₜ of about 977 ng-h/mL, a geometric mean AUC₀₋₅ of about 191 ng-h/mL, or a geometric mean AUC₅₋ₜ of about 926 ng-h/mL, as measured in children 6-12 years old under fasting conditions following dosing with the composition having the equivalent of 20 mg of amphetamine base. In certain embodiments, the pharmacokinetic profile for d-amphetamine is about 90% to about 110% of one or more of the parameters. The percentages are calculated as described herein, using a 90% confidence interval.

In certain embodiments, the extended release amphetamine solid composition provides a plasma pharmacokinetic profile for l-amphetamine which is about 80% to about 125% of one or more of one or more: a geometric mean AUCₜ of about 274 ng-h/mL, a geometric mean about AUC_{inf} of about 362 ng-h/mL, a geometric mean Cₘₐₓ of about 16 ng/mL, a geometric mean Tₘₐₓ of about 4.5, an arithmetic mean AUCₜ of about 286 ng-h/mL, an arithmetic mean about AUC_{inf} of about 380 ng-h/mL, an arithmetic mean Cₘₐₓ of about 17 ng/mL, or an arithmetic mean Tₘₐₓ of about 4.5, as measured in children 6-12 years old under fasting conditions following dosing with the composition having the equivalent of 20 mg of amphetamine base; an arithmetic mean AUC₀₋₄ of about 45 ng-h/mL, an arithmetic mean AUC₄₋ₜ of about 380 ng-h/mL, an arithmetic mean AUC₀₋₅ of about 62 ng-h/mL, an arithmetic mean AUC₅₋ₜ of about 363 ng-h/mL, a geometric mean AUC₀₋₄ of about 44 ng-h/mL, a geometric mean AUC₄₋ₜ of about 370 ng-h/mL, a geometric mean AUC₀₋₅ of about 60 ng-h/mL, or a geometric mean AUC₅₋ₜ of about 354 ng-h/mL, as measured in adults under fasting conditions following dosing with the composition having the equivalent of 20 mg of amphetamine base. The percentages are calculated as described herein, using a 90% confidence interval.

In certain embodiments, the composition used in the methods provided herein is a chewable amphetamine ER tablet. In still other embodiments, the composition is orally dissolving or dispersible ER tablet. In certain embodiments, the composition is a chewable ER tablet with is also orally dissolving, disintegrating, and/or dispersible.

The chewable ER tablet may contain a dose of about 2.5 mg to about 20 mg total amphetamines based on the amount of free amphetamine base, which is a mixture of d-amphetamine and l-amphetamine. In certain embodiments, the ratio of d-amphetamine to I-amphetamine is about 3 parts w/w d-amphetamine to about 1 part w/w l-amphetamine. This ratio may be supplied by separately combining racemic (d,l)-amphetamine and d-amphetamine, or by separately combining racemic d-amphetamine and l-amphetamine.

The ER tablet (e.g., dosage unit or dosage form) may contain about 2.5 mg amphetamines, about 5 mg amphetamine, about 15 mg amphetamines, or about 20 mg amphetamines. In certain embodiments, the tablet may be scored to allow for easy titration of the dose.

In one embodiment, the extended release amphetamine composition contains, based on the total (free base) amphetamine in the composition, at least about 15% to about 35% w/w of the amphetamine is provided by modified release component, at least about 10% to about 30% w/w of the amphetamine is provided by immediate release component comprising unbound amphetamine, and at least 40% to about 70% w/w of the amphetamine is provided by immediate release component comprising amphetamine-ion exchange resin complex. In one embodiment, each of the three components provides a ratio of d-amphetamine to l-amphetamine of about 3:about 1. In a further embodiment, the modified release component comprises 40% or less of the total amphetamines and the immediate release component comprising the amphetamine-ion exchange resin complex provides at least 60% of the total amphetamines.

In one embodiment, the composition contains only two counterions contributed by the amphetamine salts. In certain embodiments, the composition contains only one counterion contributed by the amphetamine salts.

In one embodiment, the extended release amphetamine composition provides improved driving behavior and a reduced likelihood of accidents in a subject operating a motor vehicle, and particularly, a subject having attention deficit hyperactivity disorder. In certain embodiments, a subject is dosed at least 30 minutes to 45 minutes prior to operating a motor vehicle. In certain embodiments, a subject is dosed with a single dose of an extended release amphetamine composition as provided herein within about 10 hours prior to operating or continuing to operate a motor vehicle. In certain embodiments, a patient may be administered a single dose of about 2.5 mg total amphetamines to about 20 mg total amphetamines (based on the weight of the free base form of the amphetamines) per day. However, other concentrations, e.g., of about 2.5 mg, 5 mg, 7.5 mg, 10 mg, 15 mg, 17.5 mg, or 20 mg [based on equivalence to amphetamine free base] may be readily selected although variations may occur depending upon the weight and condition of the subject being treated.

In certain subjects, the onset of improved driving behavior may be more rapid than 30 minutes or 45 minutes. Similarly, in certain subjects, the improved driving behavior may last longer than 10 hours post-dosing, e.g., up to 12 hours or up to 13 hours.

In certain embodiments, improved driving behavior is characterized by one or more of: lower crash risk, increased maximum highest braking intensity, maximum shortened braking time, increased median minimum time to collision, improved lane position and/or decreased lane deviation

In certain embodiments, the extended release amphetamine composition selected is an extended release tablet (e.g., a chewable tablet) as described herein. For example the tablet may provide a single plasma concentration peak for d-amphetamine and for l-amphetamine, wherein the tablet lacks a pH-dependent coating which provides delayed release to an amphetamine component, and wherein the tablet further comprises: (A) a modified release amphetamine component which comprises at least one modified release barrier coated amphetamine-cation exchange resin complex-optional matrix which comprises (i) two or more amphetamines bound to the same cation exchange resin, wherein when the optional matrix is present, the amphetamine-cation exchange resin complex-matrix further comprises a hydrophilic polymer or copolymer or a hydrophobic polymer and (ii) a pH-independent barrier coating which provides a modified release to the two or more amphetamines, wherein the two or more amphetamines are at least a d-amphetamine and an I-amphetamine, wherein the d-amphetamine and the l-amphetamine are provided by d-amphetamine and at least one of (d,l)-amphetamine and l-amphetamine, wherein the modified release amphetamine component (A) comprises at least 5% w/w of the total amphetamines in the tablet based on the weight of free amphetamine base; and (B) immediate release amphetamine components which comprise greater than 70% w/w of the total amphetamines in the tablet based on the total weight of free amphetamine base in the tablet, and wherein the immediate release amphetamine components further comprise: (i) a first immediate release amphetamine component which comprises d-amphetamine or a pharmaceutically acceptable salt thereof, and l-amphetamine or a pharmaceutically acceptable salt thereof, or mixtures thereof, wherein the d- and l-amphetamine are provided by d-amphetamine and at least one of (d,l)-amphetamine and l-amphetamine, wherein the immediate release amphetamine component B(i) comprises at least 5% w/w of the total amphetamines in the tablet based on the weight of free amphetamine base; (ii) amphetamine aspartate; and (iii) dextroamphetamine sulfate.

In certain embodiments, the modified release component comprises (d,l)-amphetamine and d-amphetamine. In certain embodiments, the immediate release component (C) comprises (d,l)-amphetamine and d-amphetamine. In certain embodiments, the immediate release component comprises: (a) (d,l)-amphetamine or a pharmaceutically acceptable salt thereof, and d-amphetamine or pharmaceutically acceptable salt thereof, or a mixture thereof; or (1)-amphetamine or a pharmaceutically acceptable salt thereof, (d)-amphetamine or a pharmaceutically acceptable salt thereof, or mixtures thereof, optionally further comprising (d,l)-amphetamine or a salt thereof. In certain embodiments, the tablet comprises at least two different amphetamine salts counterions. In certain embodiments, the tablet is a chewable tablet. In certain embodiments, the barrier coating is a cured, water-insoluble, water-permeable, non-ionic, pH-independent barrier coating comprises about 70 to about 90% w/w polyvinylacetate, a stabilizer, and about 2 to about 10% w/w of a plasticizer. In certain embodiments, the tablet does not exceed 500 mg in total weight.

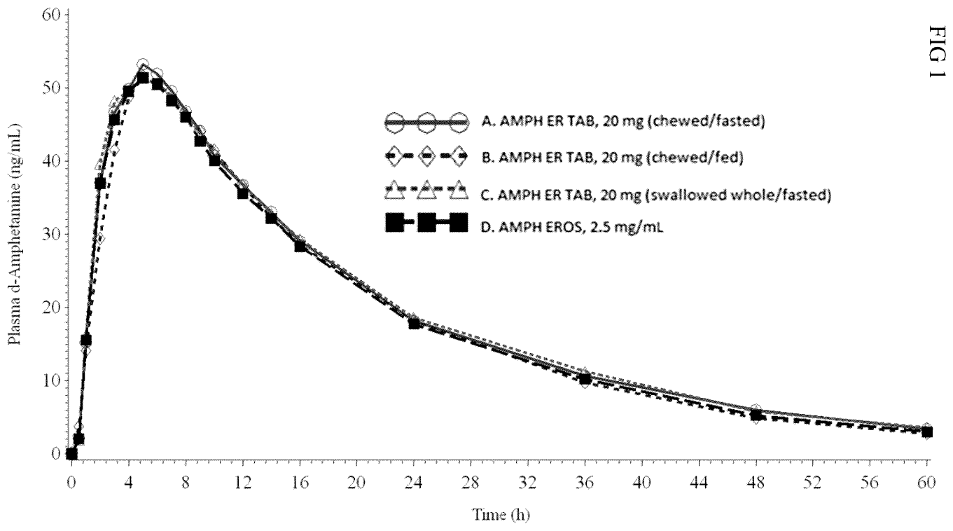

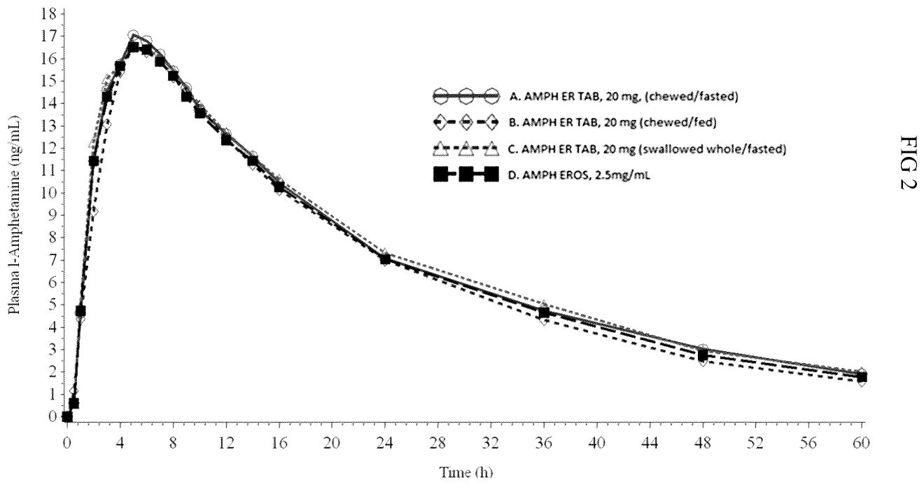

In certain embodiments, the composition affords a curve of FIG. 1 and/or FIG. 2.

In certain embodiments, the motor vehicle is an automobile.

In certain embodiments, the subject is an adult. In certain embodiments, the subject is 16 to 60 years old. In certain embodiments, the according subject is 18 to 25 years old.

In one embodiment, of the total amphetamines in the chewable tablet (based on free base of amphetamines), the modified release component contains about 15% to about 30%, or about 20% of the amphetamines, the immediate release component comprising the amphetamine-cation exchange resin complex provides about 50% to about 70%, or about 60% of the total amphetamines in the tablet, and the total unbound amphetamine is in the range of about 5% to about 25%, or about 20%, of the amphetamines. These tablets may be formulated as described previously in doses of 2.5, 5, 10, 15, 17.5 and/or 20 mg equivalent to amphetamine (based on free base weight). Other dose amounts may be selected.

Variations may nevertheless occur depending upon the weight and condition of the persons being treated and their individual responses to said medicament.

As described herein, the amphetamine extended release compositions are dosed orally once per day in the morning, e.g, before 11 am. Other variations may be selected depending upon the patient.

The following examples are illustrative only and are not intended to be a limitation on the present invention.

### EXAMPLES

The weight percentages provided in the following examples are based on the weight percentage of the total amount of the active drug in the dose using the weight of the free base equivalent, i.e., weight on the active drug calculated or measured without the weight of the coating, matrix polymer, polistirex, counterion, or hydrate form of the compound.

### Example A: Uncoated ((d,dl-)Amphetamine-Cation Exchange Resin Complex

| | |
|---|---|
| Ingredients | Quantity |

Uncoated (d, dl) Amphetamine - Cation Exchange Resin Complex

| | | |
|---|---|---|
| Sodium Polystyrene Sulfonate (Amberlite | 12000 | g |
| IRP69 ®) Cation Exchange Resin | | |
| (dl) Amphetamine Sulfate | 1440 | g |
| (d)-Amphetamine Sulfate | 1560 | g |
| Purified Water | 70 | L |

The Drug-Resin Complex is prepared by first adding 70 L of Purified Water into the process vessel, following which (dl)-Amphetamine Sulfate and (d)-Amphetamine Sulfate are dissolved by continuous mixing. Sodium Polystyrene Sulfonate is dispersed with continuous mixing and the mixing is continued for 60 minutes. Water is removed by filtration process followed by rinsing twice using purified water (40 L). Wet resin complex is then dried until moisture content is between 3% to 7%. Dried drug-resin complex is passed through a 40 mesh screen using the CO-MIL. This is the Uncoated (d,dl) amphetamine-cation exchanger resin complex, which is used directly in the compositions described beginning with Example 1 and for preparing the Uncoated (d,dl) amphetamine-cation exchanger resin complex-matrix, which is subsequently coated.

### Example B: Uncoated ((d,dl-)Amphetamine-Cation Exchange Resin Complex Matrix

| | |
|---|---|
| Ingredients | Quantity |

(d, dl) Amphetamine - Cation Exchange Resin - Matrix

| | | |
|---|---|---|
| Uncoated (d, dl) Amphetamine - Cation | 10000 | g |
| Exchange Resin Complex | | |
| Povidone (Kollidon ® 30D) | 500 | g |
| Purified water | 3500 | g |

In a separate container Povidone is dissolved in 3500 g of Purified Water (Povidone solution). Uncoated (d,dl) Amphetamine-cation exchange resin complex prepared as described in Example A is treated with Povidone solution with continuous mixing to form a uniform mass. Wet mass is dried until the moisture content is between 15-25%. Semi-dried material is then milled using CO-MIL brand mill fitted with 40 mesh screen. Milled material is further dried until moisture content is between 3-7%. Dried material is passed through a CO-MIL fitted with a 40 mesh screen. This is the (d,dl) amphetamine-cation exchange resin complex-matrix.

### Example C: Barrier Coated ((d, dl-)Amphetamine-Cation Exchange Resin Complex-Matrix, with Polyvinylacetate-Plasticizer Barrier Coating

1. Coated (d,dl) Amphetamine-Cation Exchange Resin with 30% Weight Gain

| | |
|---|---|
| Ingredients | Quantity |

Coated (d, dl) Amphetamine - Cation Exchange resin
Complex - matrix

| | | |
|---|---|---|
| (d, dl) Amphetamine - Cation Exchange Resin | 2500 | g |
| Complex - Matrix | | |
| Polyvinyl Acetate Dispersion** (Kollicoat ® | 3500 | g |
| SR30D) | | |
| Triacetin | 52.5 | g |
| Purified Water | 1960 | g |

Removed during processing
**30% w/w aqueous dispersion

The Coated (d,dl) Amphetamine-cation exchange resin complex-matrix is prepared as follows. The coating solution is prepared by mixing Triacetin, Purified Water and Polyvinyl Acetate dispersion in a separate container. The coating process is performed in a fluid bed processor equipped with Wurster column by applying coating solution on to (d,dl) Amphetamine-cation exchange resin complex-matrix as prepared in Example B, that results in 30% weight gain. The Coated (d,dl) Amphetamine-Cation Exchange Resin complex-matrix is placed in the hot air oven at 60° C. for 5 hours. The Coated (d,dl) Amphetamine-Cation exchange resin complex-Matrix is again passed through Sieve No. 40 mesh screen. This is the Coated (d,dl) Amphetamine-Cation Exchange Resin Complex-Matrix.

2. Coated (d,dl) Amphetamine-Cation Exchange Resin with 40% Weight Gain

Coated (d,dl) Amphetamine-cation exchange resin complex is prepared as follows. The coating solution is prepared by mixing Triacetin, Purified Water and Polyvinylacetate dispersion in a separate container. The coating process is performed in a fluid bed processor equipped with Wurster column by applying adequate quantity of the coating solution on to uncoated (d,dl) Amphetamine-cation exchange resin complex as prepared in Example B, that results in 40% weight gain. The Coated (d,dl) Amphetamine-Cation Exchange Resin complex is placed in the hot air oven at 60° C. for 5 hours. The Coated (d,dl) Amphetamine-Cation exchange resin complex is passed through Sieve No. 40 mesh screen. This is the Coated (d,dl) Amphetamine-Cation Exchange Resin.

| | |
|---|---|
| Ingredients | Quantity |

Coated (d, dl) Amphetamine - Cation Exchange resin
Complex - matrix

| | | |
|---|---|---|
| (d, dl) Amphetamine - Cation Exchange Resin | 600 | g |
| Complex - Matrix | | |
| Polyvinyl Acetate Dispersion** (Kollicoat ® | 1015 | g |
| SR30D) | | |
| Triacetin | 15 | g |
| Purified Water | 570 | g |

3. Coated (d,dl) Amphetamine-Cation Exchange Resin with 50% Weight Gain

Coated (d,dl) Amphetamine-cation exchange resin complex with no matrix is prepared as follows. The coating solution is prepared by mixing Triacetin, Purified Water and Polyvinyl Acetate dispersion in a separate container. The coating process is performed in a fluid bed processor equipped with Wurster column by applying adequate quantity of the coating solution on to uncoated (d,dl) Amphetamine-cation exchange resin complex as prepared in Example B, that results in 50% weight gain. The Coated (d,dl) Amphetamine-Cation Exchange Resin complex is placed in the hot air oven at 60° C. for 5 hours. The Coated (d,dl) Amphetamine-Cation exchange resin complex is passed through Sieve No. 40 mesh screen. This is the Coated (d,dl) Amphetamine-Cation Exchange Resin.

| | |
|---|---|
| Ingredients | Quantity |

Coated (d, dl) Amphetamine - Cation Exchange resin
Complex - matrix

| | | |
|---|---|---|
| (d, dl) Amphetamine - Cation Exchange Resin | 600 | g |
| Complex - Matrix | | |
| Polyvinyl Acetate Dispersion** (Kollicoat ® | 1015 | g |
| SR30D) | | |
| Triacetin | 15 | g |
| Purified Water | 570 | g |

### Example D: Barrier Coated ((d, dl-)Amphetamine-Cation Exchange Resin Complex-Matrix, with Ethylcellulose Barrier Coating

### 1. Aqueous Ethylcellulose

| | |
|---|---|
| Ingredients | Quantity |

Coated (d, dl) Amphetamine - Cation Exchange resin
Complex - matrix

| | | |
|---|---|---|
| (d, dl) Amphetamine - Cation Exchange Resin | 2500 | g |
| Complex - Matrix | | |
| Ethylcellulose (Surerelease ®) | 3500 | g |
| Triacetin | 52.5 | g |
| Purified Water | 1960 | g |

Coated (d,dl) Amphetamine-cation exchange resin complex is prepared as follows. The coating solution is prepared by mixing Triacetin, Purified Water and Ethylcellulose dispersion in a separate container. The coating process is performed in a fluid bed processor equipped with Wurster column by applying coating solution on to uncoated (d,dl) Amphetamine-cation exchange resin complex as prepared in Example B, that results in 30% weight gain. The Coated (d,dl) Amphetamine-Cation Exchange Resin complex is placed in the hot air oven at 60° C. for 5 hours. The Coated (d,dl) Amphetamine-Cation exchange resin complex is passed through Sieve No. 40 mesh screen. This is the Coated (d,dl) Amphetamine-Cation Exchange Resin.

### 2. Solvent-Based Ethylcellulose

| | |
|---|---|
| Ingredients | Quantity |

Coated (d, dl) Amphetamine - Cation Exchange resin
Complex - matrix

| | | |
|---|---|---|
| (d, dl) Amphetamine - Cation Exchange Resin | 2500 | g |
| Complex - Matrix | | |
| Ethylcellulose (Ethocel ®) | 3500 | g |
| Triacetin | 52.5 | g |
| Ethanol/water | 1960 | g |

Coated (d,dl) Amphetamine-cation exchange resin complex is prepared as follows. The coating solution is prepared by mixing Triacetin, Purified Water and Ethylcellulose solution in a separate container. The coating process is performed in a fluid bed processor equipped with Wurster column by applying coating solution on to uncoated (d,dl) Amphetamine-cation exchange resin complex as prepared in Example B, that results in 30% weight gain. The Coated (d,dl) Amphetamine-Cation Exchange Resin complex is placed in the hot air oven at 60° C. for 5 hours. The Coated (d,dl) Amphetamine-Cation exchange resin complex is passed through Sieve No. 40 mesh screen. This is the Coated (d,dl) Amphetamine-Cation Exchange Resin.

### Example E: Barrier Coated ((d, dl-)Amphetamine-Cation Exchange Resin Complex-Matrix, with pH-Independent Eudragit® RL30D Polyacrylic Aqueous Dispersion—Barrier Coating

| | |
|---|---|
| Ingredients | Quantity |

Coated (d, dl) Amphetamine - Cation Exchange resin
Complex - matrix

| | | |
|---|---|---|
| (d, dl) Amphetamine - Cation Exchange Resin | 2500 | g |
| Complex - Matrix | | |
| Eudragit ® RL30D Polyacrylic aqueous | 3500 | g |
| dispersion | | |
| Triacetin | 52.5 | g |
| Purified Water | 1960 | g |

Coated (d,dl) Amphetamine-cation exchange resin complex is prepared as follows. The coating solution is prepared by mixing Triacetin, Purified Water and Eudragit® RL30D Polyacrylic aqueous dispersion in a separate container. The coating process is performed in a fluid bed processor equipped with Wurster column by applying coating solution on to uncoated (d,dl) Amphetamine-cation exchange resin complex as prepared in Example B, that results in 30% weight gain. The Coated (d,dl) Amphetamine-Cation Exchange Resin complex is placed in the hot air oven at 60° C. for 5 hours. The Coated (d,dl) Amphetamine-Cation exchange resin complex is passed through Sieve No. 40 mesh screen. This is the Coated (d,dl) Amphetamine-Cation Exchange Resin.

### Example F: Barrier Coated ((d,dl-)Amphetamine-Cation Exchange Resin Complex-Matrix, with pH-independent Cellulose Acetate—Barrier Coating

1. The Coated (d,dl) Amphetamine-Cation Exchange Resin Complex Matrix.

| | |
|---|---|
| Ingredients | Quantity |

Coated (d, dl) Amphetamine - Cation Exchange resin
Complex - matrix

| | | |
|---|---|---|
| (d, dl) Amphetamine - Cation Exchange Resin | 2500 | g |
| Complex - Matrix | | |
| Cellulose Acetate (CA-398-10 NF/EP) | 850 | g |
| Polyethylene Glycol | 200 | g |
| Triacetin | 52.5 | g |
| Acetone/Water (80/20 wt %) ** | 2000 | g |

** Removed during processing

The Coated (d,dl) Amphetamine-cation exchange resin complex matrix is prepared as follows. The coating solution is prepared by mixing the cellulose acetate and polyethylene glycol in acetone/water until completely dissolved, following by mixing with triacetin in a separate container. The coating process is performed in a fluid bed processor equipped with Wurster column by applying coating solution on to (d,dl) Amphetamine-cation exchange resin complex-matrix as prepared in Example B, that results in 25% weight gain. The Coated (d,dl) Amphetamine-Cation Exchange Resin complex-matrix is placed in the hot air oven at 60° C. for 5 hours. The Coated (d,dl) Amphetamine-Cation exchange resin complex-matrix is again passed through Sieve No. 40 mesh screen. This is the Coated (d,dl) Amphetamine-Cation Exchange Resin Complex-Matrix.

2. The Coated (d,dl) Amphetamine-Cation Exchange Resin Complex

| | |
|---|---|
| Ingredients | Quantity |

Coated (d, dl) Amphetamine - Cation Exchange resin
Complex

| | | |
|---|---|---|
| (d, dl) Amphetamine - Cation Exchange Resin | 2500 | g |
| Complex | | |
| Cellulose Acetate (CA-398-10 NF/EP) | 850 | g |
| Polyethylene Glycol | 200 | g |
| Triacetin | 52.5 | g |
| Acetone/Water (80/20%) ** | 2000 | g |

The Coated (d,dl) Amphetamine-cation exchange resin complex is prepared as follows. The coating solution is prepared by mixing the cellulose acetate and polyethylene glycol in acetone/water until completely dissolved, following by mixing with triacetin in a separate container. The coating process is performed in a fluid bed processor equipped with Wurster column by applying coating solution on to (d,dl) Amphetamine-cation exchange resin complex as prepared in Example A, that results in 25% weight gain. The Coated (d,dl) Amphetamine-Cation Exchange Resin complex is placed in the hot air oven at 60° C. for 5 hours. The Coated (d,dl) Amphetamine-Cation exchange resin complex is again passed through Sieve No. 40 mesh screen. This is the Coated (d,dl) Amphetamine-Cation Exchange Resin Complex.

In the following examples, chewable tablets and methods of making same are illustrated.

### Example 1: Amphetamine Extended Release (ER) Chewable Tablets Equivalent to 20 mg Amphetamine Free Base, with Polyvinylacetate Barrier Coated Modified Release Component (30/50/20—Coated/Uncoated/API)

In the following study, a polyvinylacetate-plasticizer barrier coating of Example C1 (30% weight gain) is selected for the Barrier Coated Amphetamine-cation exchange resin complex-matrix.

| Ingredients | % w/w |
|---|---|
| Barrier Coated Amphetamine - cation exchange resin | 6.881 |
| complex - matrix | |
| Uncoated Amphetamine - cation exchange resin complex | 7.998 |
| Dextroamphetamine Sulfate, USP | 0.356 |
| Amphetamine Aspartate Monohydrate | 0.511 |
| Mannitol, USP | 53.054 |
| Talc, USP | 1.0 |
| Xanthan gum, NF (Gumixan ™ K) | 0.5 |
| Crospovidone, NF (Kollidon ® CL), Type A | 12 |
| Microcrystalline cellulose and Guargum (Avicel ® CE | 15 |
| Sucralose, NF | 1.5 |
| Bubblegum Flavor | 0.5 |
| Silicon dioxide, NF (Syloid ® 244FP) | 0.2 |
| Magnesium stearate, NF | 0.5 |
| Total | 100.0 |

The extended release chewable tablets containing amphetamines are prepared as follows. Weigh the mannitol and microcrystalline cellulose and guar gum (Avicel CE15), and screen through a 20 mesh screen. Add the screened material into ‘V’-Blender. The xanthan gum, Kollidon® CL, sucralose, citric acid, bubble gum flavor, silicon dioxide, talc, Uncoated Amphetamine-cation exchange resin complex of Example A, Coated Amphetamine-cation exchange resin complex-matrix, Dextroamphetamine Sulfate and Amphetamine Aspartate Monohydrate are weighed and passed through a #40 mesh screen. The screened material is added into a ‘V’-Blender. Mix the powder blend for 15 minutes. Weigh Magnesium Stearate and pass through #40 mesh screen. Add the screened material into ‘V’-Blender. Mix this powder blend for 10 minutes. The material from the Blender is unloaded and compressed into tablets by adjusting the appropriate tablet weight and hardness.

### Example 2—Amphetamine Extended Release (ER) Chewable Tablets Equivalent to 20 mg Amphetamine Free Base, with Ethylcellulose Barrier Coated Modified Release Component (30/50/20—Coated/Uncoated/API)

In the following study, an aqueous ethylcellulose barrier coating of Example D1 (30% weight gain) is selected for the Barrier Coated Amphetamine-cation exchange resin complex-matrix. In other embodiments, the solvent-based ethylcellulose barrier coating of example D2 is selected for the Barrier Coated Amphetamine-cation exchange resin complex-matrix.

| Ingredients | % w/w |
|---|---|
| Barrier Coated Amphetamine - cation | 6.881 |
| exchange resin complex - matrix | |
| Uncoated Amphetamine - cation | 7.998 |
| exchange resin complex | |
| Dextroamphetamine Sulfate, USP | 0.356 |
| Amphetamine Aspartate Monohydrate | 0.511 |
| Mannitol, USP | 53.054 |
| Talc, USP | 1.0 |
| Xanthan gum, NF (Gumixan ™ K) | 0.5 |
| Crospovidone, NF (Kollidon ® CL), | 12 |
| Type A | |
| Microcrystalline cellulose and | 15 |
| Guargum (Avicel ® CE | |
| Sucralose, NF | 1.5 |
| Bubblegum Flavor | 0.5 |
| Silicon dioxide, NF (Syloid ® 244FP) | 0.2 |
| Magnesium stearate, NF | 0.5 |
| Total | 100.0 |

The extended release chewable tablets containing amphetamines are prepared as follows. Weigh the mannitol and microcrystalline cellulose and guar gum (Avicel CE15), and screen through a 20 mesh screen. Add the screened material into ‘V’-Blender. The xanthan gum, Kollidon® CL, sucralose, citric acid, bubble gum flavor, silicon dioxide, talc, Uncoated Amphetamine-cation exchange resin complex of Example A, Coated Amphetamine-cation exchange resin complex-matrix, Dextroamphetamine Sulfate and Amphetamine Aspartate Monohydrate are weighed and passed through a #40 mesh screen. The screened material is added into a ‘V’-Blender. Mix the powder blend for 15 minutes. Weigh Magnesium Stearate and pass through #40 mesh screen. Add the screened material into ‘V’-Blender. Mix this powder blend for 10 minutes. The material from the Blender is unloaded and compressed into tablets by adjusting the appropriate tablet weight and hardness.

### Example 3—Amphetamine Extended Release (ER) Chewable Tablets Equivalent to 20 mg Amphetamine Free Base, with Cellulose Acetate Barrier Coated Modified Release Component (30/50/20—Coated/Uncoated/API)

In the following study, a cellulose acetate barrier coating of Example F1 (25% weight gain) is selected for the Barrier Coated Amphetamine-cation exchange resin complex-matrix. In alternative embodiments, the cellulose acetate barrier coating of Example F2 is selected for the Barrier coated Amphetamine-cation exchange resin complex (coating over AMP-cation exchange resin complex of Example A).

| Ingredients | % w/w |
|---|---|
| Barrier Coated Amphetamine - cation exchange | 6.881 |
| resin complex or complex-matrix | |
| Uncoated Amphetamine - cation exchange | 7.998 |
| resin complex | |
| Dextroamphetamine Sulfate, USP | 0.356 |
| Amphetamine Aspartate Monohydrate | 0.511 |
| Mannitol, USP | 53.054 |
| Talc, USP | 1.0 |
| Xanthan gum, NF (Gumixan ™ K) | 0.5 |
| Crospovidone, NF (Kollidon ® CL), Type A | 12 |
| Microcrystalline cellulose and | 15 |
| Guargum (Avicel ® CE 15) | |
| Sucralose, NF | 1.5 |
| Bubblegum Flavor | 0.5 |
| Silicon dioxide, NF (Syloid ® 244FP) | 0.2 |
| Magnesium stearate, NF | 0.5 |
| Total | 100.0 |

The extended release chewable tablets containing amphetamines are prepared as follows. Weigh the mannitol and microcrystalline cellulose and guar gum (Avicel CE15), and screen through a 20 mesh screen. Add the screened material into ‘V’-Blender. The xanthan gum, Kollidon® CL, sucralose, citric acid, bubble gum flavor, silicon dioxide, talc, Uncoated Amphetamine-cation exchange resin complex of Example A, Coated Amphetamine-cation exchange resin complex-matrix, Dextroamphetamine Sulfate and Amphetamine Aspartate Monohydrate are weighed and passed through a #40 mesh screen. The screened material is added into a ‘V’-Blender. Mix the powder blend for 15 minutes. Weigh Magnesium Stearate and pass through #40 mesh screen. Add the screened material into ‘V’-Blender. Mix this powder blend for 10 minutes. The material from the Blender is unloaded and compressed into tablets by adjusting the appropriate tablet weight and hardness.

### Example 4—Amphetamine Extended Release (ER) Chewable Tablets Equivalent to 20 mg Amphetamine Free Base, with Eudragit Barrier Coated Modified Release Component (30/50/20—Coated/Uncoated/API)

In the following study, an acrylate based barrier coating of Example E (3000 weight gain) is selected for the Barrier Coated Amphetamine-cation exchange resin complex-matrix.

| Ingredients | % w/w |
|---|---|
| Barrier Coated Amphetamine - cation | 6.881 |
| exchange resin complex | |
| Uncoated Amphetamine - cation | 7.998 |
| exchange resin complex | |
| Dextroamphetamine Sulfate, USP | 0.356 |
| Amphetamine Aspartate Monohydrate | 0.511 |
| Mannitol, USP | 53.054 |
| Talc, USP | 1.0 |
| Xanthan gum, NF (Gumixan ™ K) | 0.5 |
| Crospovidone, NF (Kollidon ® CL), | 12 |
| Type A | |
| Microcrystalline cellulose and | 15 |
| Guargum (Avicel ® CE | |
| Sucralose, NF | 1.5 |
| Bubblegum Flavor | 0.5 |
| Silicon dioxide, NF (Syloid ® 244FP) | 0.2 |
| Magnesium stearate, NF | 0.5 |
| Total | 100.0 |

The extended release chewable tablets containing amphetamines are prepared as follows. Weigh the mannitol and microcrystalline cellulose and guar gum (Avicel CE15), and screen through a 20 mesh screen. Add the screened material into ‘V’-Blender. The xanthan gum, Kollidon® CL, sucralose, citric acid, bubble gum flavor, silicon dioxide, talc, Uncoated Amphetamine-cation exchange resin complex of Example A, Coated Amphetamine-cation exchange resin complex-matrix of Example E, Dextroamphetamine Sulfate and Amphetamine Aspartate Monohydrate are weighed and passed through a #40 mesh screen. The screened material is added into a ‘V’-Blender Mix the powder blend for 15 minutes. Weigh Magnesium Stearate and pass through #40 mesh screen. Add the screened material into ‘V’-Blender. Mix this powder blend for 10 minutes. The material from the Blender is unloaded and compressed into tablets by adjusting the appropriate tablet weight and hardness.

### Example 5—Amphetamine Extended Release (ER) Chewable Tablets Equivalent to 20 mg Amphetamine Free Base

In the following study, a polyvinylacetate barrier coating of Example C1 (30% weight gain) is selected for the Barrier Coated Amphetamine-cation exchange resin complex-matrix.

| Ingredients | % w/w |
|---|---|
| Barrier Coated Amphetamine - cation | 6.881 |
| exchange resin complex - matrix | |
| Uncoated Amphetamine - cation | 7.998 |
| exchange resin complex | |
| Dextroamphetamine Saccharate | 0.356 |
| Amphetamine sulfate | 0.511 |
| Mannitol, USP | 53.054 |
| Talc, USP | 1.0 |
| Xanthan gum, NF (Gumixan ™ K) | 0.5 |
| Crospovidone, NF (Kollidon ® CL), | 12 |
| Type A | |
| Microcrystalline cellulose and | 15 |
| Guargum (Avicel ® CE | |
| Sucralose, NF | 1.5 |
| Bubblegum Flavor | 0.5 |
| Silicon dioxide, NF (Syloid 244FP) | 0.2 |
| Magnesium stearate, NF | 0.5 |
| Total | 100.0 |

The extended release chewable tablets containing amphetamines are prepared as follows. Weigh the mannitol and microcrystalline cellulose and guar gum (Avicel CE15), and screen through a 20 mesh screen. Add the screened material into ‘V’-Blender. The xanthan gum, Kollidon® CL, sucralose, citric acid, bubble gum flavor, silicon dioxide, talc, Uncoated Amphetamine-cation exchange resin complex, Coated Amphetamine-cation exchange resin complex-matrix, Dextroamphetamine Saccharate and Amphetamine Sulfate are weighed and passed through a #40 mesh screen. The screened material is added into a ‘V’-Blender. Mix the powder blend for 15 minutes. Weigh Magnesium Stearate and pass through #40 mesh screen. Add the screened material into ‘V’-Blender. Mix this powder blend for 10 minutes. The material from the Blender is unloaded and compressed into tablets by adjusting the appropriate tablet weight and hardness.

### Example 6—Amphetamine Extended Release (ER) Chewable Tablets Equivalent to 20 mg Amphetamine Free Base 20/60/20—Coated/Uncoated/API

In the following study, an ethylcellulose barrier coating of Example C1 (30% weight gain) is selected for the Barrier Coated Amphetamine-cation exchange resin complex-matrix.

| Ingredients | % w/w |
|---|---|
| Barrier Coated Amphetamine - cation | 4.583 |
| exchange resin complex - matrix | |
| Uncoated Amphetamine - cation | 9.68 |
| exchange resin complex | |
| Dextroamphetamine Sulfate, USP | 0.356 |
| Amphetamine Aspartate Monohydrate | 0.511 |
| Mannitol, USP | 53.67 |
| Xanthan gum, NF (Gumixan ™ K) | 0.5 |
| Crospovidone, NF (Kollidon ® CL), | 12 |
| Type A | |
| Microcrystalline cellulose and | 15 |
| Guargum (Avicel ® CE | |
| Sucralose, NF | 1.5 |
| Bubblegum Flavor | 0.5 |
| Silicon dioxide, NF (Syloid ® 244FP) | 0.2 |
| Talc, USP | 1.0 |
| Magnesium stearate, NF | 0.5 |
| Total | 100.0 |

The extended release chewable tablets containing amphetamines are prepared as follows. Weigh the mannitol and microcrystalline cellulose and guar gum (Avicel® CE15), and screen through a 20 mesh screen. Add the screened material into ‘V’-Blender. The xanthan gum, Kollidon® CL, sucralose, citric acid, bubble gum flavor, silicon dioxide, talc, Uncoated Amphetamine-cation exchange resin complex of Example A, Coated Amphetamine-cation exchange resin complex-matrix, Dextroamphetamine Sulfate and Amphetamine Aspartate Monohydrate are weighed and passed through a #40 mesh screen. The screened material is added into a ‘V’-Blender. Mix the powder blend for 25 minutes. Weigh Magnesium Stearate and pass through #40 mesh screen. Add the screened material into ‘V’-Blender. Mix this powder blend for 5 minutes. The material from the Blender is unloaded and compressed into tablets by adjusting the appropriate tablet weight and hardness.

### Example 7—Amphetamine Extended Release (ER) Chewable Tablets Equivalent to 20 mg Amphetamine Free Base

In the following study, an ethylcellulose barrier coating of Example D1 (30% weight gain) is selected for the Barrier Coated Amphetamine-cation exchange resin complex-matrix.

| Ingredients | % w/w |
|---|---|
| Barrier Coated Amphetamine - cation | 4.583 |
| exchange resin complex - matrix | |
| Uncoated Amphetamine - cation | 9.68 |
| exchange resin complex | |
| Dextroamphetamine Saccharate | 0.356 |
| Amphetamine sulfate | 0.511 |
| Mannitol, USP | 53.67 |
| Xanthan gum, NF (Gumixan ™ K) | 0.5 |
| Crospovidone, NF (Kollidon ® CL), | 12 |
| Type A | |
| Microcrystalline cellulose and | 15 |
| Guargum (Avicel ® CE | |
| Sucralose, NF | 1.5 |
| Bubblegum Flavor | 0.5 |
| Silicon dioxide, NF (Syloid ® 244FP) | 0.2 |
| Talc, USP | 1.0 |
| Magnesium stearate, NF | 0.5 |
| Total | 100.0 |

The extended release chewable tablets containing amphetamines are prepared as follows. Weigh the mannitol and microcrystalline cellulose and guar gum (Avicel® CE15), and screen through a 20 mesh screen. Add the screened material into ‘V’-Blender. The xanthan gum, Kollidon® CL, sucralose, citric acid, bubble gum flavor, silicon dioxide, talc, Uncoated Amphetamine-cation exchange resin complex of Example A, Coated Amphetamine-cation exchange resin complex-matrix, Dextroamphetamine Saccharate and Amphetamine sulfate are weighed and passed through a #40 mesh screen. The screened material is added into a ‘V’-Blender. Mix the powder blend for 25 minutes. Weigh Magnesium Stearate and pass through #40 mesh screen. Add the screened material into ‘V’-Blender. Mix this powder blend for 5 minutes. The material from the Blender is unloaded and compressed into tablets by adjusting the appropriate tablet weight and hardness.

### Example 8—Amphetamine Extended Release (ER) Chewable Tablets Equivalent to 20 mg Amphetamine Free Base

In the following study, an ethylcellulose barrier coating of Example D1 (30% weight gain) is selected for the Barrier Coated Amphetamine-cation exchange resin complex-matrix.

| Ingredients | % w/w |
|---|---|
| Barrier Coated Amphetamine - cation | 4.583 |
| exchange resin complex - matrix | |
| Uncoated Amphetamine - cation | 9.68 |
| exchange resin complex | |
| Dextroamphetamine Sulfate, USP | 0.356 |
| Amphetamine Aspartate Monohydrate | 0.511 |
| Mannitol, USP | 53.67 |
| Xanthan gum, NF (Gumixan ™ K) | 0.5 |
| Crospovidone, NF (Kollidon ® CL), | 12 |
| Type A | |
| Microcrystalline cellulose and | 15 |
| Guargum (Avicel ® CE | |
| Sucralose, NF | 1.5 |
| Bubblegum Flavor | 0.5 |
| Silicon dioxide, NF (Syloid ® 244FP) | 0.2 |
| Talc, USP | 1.0 |
| Magnesium stearate, NF | 0.5 |
| Total | 100.0 |

The extended release chewable tablets containing amphetamines are prepared as follows. Weigh the mannitol and microcrystalline cellulose and guar gum (Avicel CE15), and screen through a 20 mesh screen. Add the screened material into ‘V’-Blender. The xanthan gum, Kollidon® CL, sucralose, citric acid, bubble gum flavor, silicon dioxide, talc, Uncoated Amphetamine-cation exchange resin complex of Example A, Coated Amphetamine-cation exchange resin complex-matrix, Dextroamphetamine Sulfate and Amphetamine Aspartate Monohydrate are weighed and passed through a #40 mesh screen. The screened material is added into a ‘V’-Blender Mix the powder blend for 25 minutes. Weigh Magnesium Stearate and pass through #40 mesh screen. Add the screened material into ‘V’-Blender. Mix this powder blend for 5 minutes. The material from the Blender is unloaded and compressed into tablets by adjusting the appropriate tablet weight and hardness.

### Example 9—Amphetamine Extended Release (ER) Chewable Tablets Equivalent to 20 mg Amphetamine Free Base

In the following study, a cellulose acetate coating of Example F (2500 weight gain) is selected for the Barrier Coated Amphetamine-cation exchange resin complex-matrix.

| Ingredients | % w/w |
|---|---|
| Barrier Coated Amphetamine - cation | 4.583 |
| exchange resin complex - matrix | |
| Uncoated Amphetamine - cation | 9.68 |
| exchange resin complex | |
| Dextroamphetamine Sulfate, USP | 0.356 |
| Amphetamine Aspartate Monohydrate | 0.511 |
| Mannitol, USP | 53.67 |
| Xanthan gum, NF (Gumixan ™ K) | 0.5 |
| Crospovidone, NF (Kollidon ® CL), | 12 |
| Type A | |
| Microcrystalline cellulose and | 15 |
| Guargum (Avicel ® CE | |
| Sucralose, NF | 1.5 |
| Bubblegum Flavor | 0.5 |
| Silicon dioxide, NF (Syloid ® 244FP) | 0.2 |
| Talc, USP | 1.0 |
| Magnesium stearate, NF | 0.5 |
| Total | 100.0 |

The extended release chewable tablets containing amphetamines are prepared as follows. Weigh the mannitol and microcrystalline cellulose and guar gum (Avicel CE15), and screen through a 20 mesh screen. Add the screened material into ‘V’-Blender. The xanthan gum, Kollidon® CL, sucralose, citric acid, bubble gum flavor, silicon dioxide, talc, Uncoated Amphetamine-cation exchange resin complex of Example A, Coated Amphetamine-cation exchange resin complex-matrix, Dextroamphetamine Sulfate and Amphetamine Aspartate Monohydrate are weighed and passed through a #40 mesh screen. The screened material is added into a ‘V’-Blender. Mix the powder blend for 25 minutes. Weigh Magnesium Stearate and pass through #40 mesh screen. Add the screened material into ‘V’-Blender. Mix this powder blend for 5 minutes. The material from the Blender is unloaded and compressed into tablets by adjusting the appropriate tablet weight and hardness.

### Example 10—Amphetamine Extended Release (ER) Chewable Tablets Equivalent to 20 mg Amphetamine Free Base

In the following study, an acrylate based barrier coating of Example E (30% weight gain) is selected for the Barrier Coated Amphetamine-cation exchange resin complex-matrix.

| Ingredients | % w/w |
|---|---|
| Barrier Coated Amphetamine - cation | 4.583 |
| exchange resin complex - matrix | |
| Uncoated Amphetamine - cation | 9.68 |
| exchange resin complex | |
| Dextroamphetamine Sulfate, USP | 0.356 |
| Amphetamine Aspartate Monohydrate | 0.511 |
| Mannitol, USP | 53.67 |
| Xanthan gum, NF (Gumixan ™ K) | 0.5 |
| Crospovidone, NF (Kollidon ® CL), | 12 |
| Type A | |
| Microcrystalline cellulose and | 15 |
| Guargum (Avicel ® CE | |
| Sucralose, NF | 1.5 |
| Bubblegum Flavor | 0.5 |
| Silicon dioxide, NF (Syloid ® 244FP) | 0.2 |
| Talc, USP | 1.0 |
| Magnesium stearate, NF | 0.5 |
| Total | 100.0 |

The extended release chewable tablets containing amphetamines are prepared as follows. Weigh the mannitol and microcrystalline cellulose and guar gum (Avicel CE15), and screen through a 20 mesh screen. Add the screened material into ‘V’-Blender. The xanthan gum, Kollidon® CL, sucralose, citric acid, bubble gum flavor, silicon dioxide, talc, Uncoated Amphetamine-cation exchange resin complex of Example A, Coated Amphetamine-cation exchange resin complex-matrix, Dextroamphetamine Sulfate and Amphetamine Aspartate Monohydrate are weighed and passed through a #40 mesh screen. The screened material is added into a ‘V’-Blender. Mix the powder blend for 25 minutes. Weigh Magnesium Stearate and pass through #40 mesh screen. Add the screened material into ‘V’-Blender. Mix this powder blend for 5 minutes. The material from the Blender is unloaded and compressed into tablets by adjusting the appropriate tablet weight and hardness.

### Example 11—Amphetamine Extended Release (ER) Chewable Tablets Equivalent to 10 mg Amphetamine Free Base

In the following study, a polyvinylacetate barrier coating of Example C1 (30% weight gain) is selected for the Barrier Coated Amphetamine-cation exchange resin complex-matrix.

| Ingredients | % w/w |
|---|---|
| Barrier Coated Amphetamine - cation | 2.29 |
| exchange resin complex - matrix ¹ | |
| Uncoated Amphetamine - cation | 4.84 |
| exchange resin complex ¹ | |
| Dextroamphetamine Sulfate, USP | 0.178 |
| Amphetamine Aspartate Monohydrate² | 0.255 |
| Mannitol, USP ³ | 61.23 |
| Xanthan gum, NF (Gumixan ™ K) | 0.5 |
| Crospovidone, NF (Kollidon ® CL), | 12 |
| Type A | |
| Microcrystalline cellulose and | 15 |
| Guargum (Avicel ® CE | |
| Sucralose, NF | 1.5 |
| Bubblegum Flavor | 0.5 |
| Silicon dioxide, NF (Syloid ® 244FP) | 0.2 |
| Talc, USP | 1.0 |
| Magnesium stearate, NF | 0.5 |
| Total | 100.0 |

The extended release chewable tablets containing amphetamines are prepared as follows. Weigh the mannitol and microcrystalline cellulose and guar gum (Avicel CE15), and screen through a 20 mesh screen. Add the screened material into ‘V’-Blender. The xanthan gum, Kollidon® CL, sucralose, citric acid, bubble gum flavor, silicon dioxide, talc, Uncoated Amphetamine-cation exchange resin complex of Example A, Coated Amphetamine-cation exchange resin complex-matrix, Dextroamphetamine Sulfate and Amphetamine Aspartate Monohydrate are weighed and passed through a #40 mesh screen. The screened material is added into a ‘V’-Blender. Mix the powder blend for 25 minutes. Weigh Magnesium Stearate and pass through #40 mesh screen. Add the screened material into ‘V’-Blender. Mix this powder blend for 5 minutes. The material from the Blender is unloaded and compressed into tablets by adjusting the appropriate tablet weight and hardness.

### Example 12—Amphetamine Extended Release (ER) Chewable Tablets Equivalent to 5 mg Amphetamine Free Base

In the following study, a polyvinyl acetate barrier coating of Example C1 (3000 weight gain) is selected for the Coated Amphetamine-cation exchange resin complex-matrix.

| Ingredients | % w/w |
|---|---|
| Barrier Coated Amphetamine - cation | 1.72 |
| exchange resin complex - matrix ¹ | |
| Uncoated Amphetamine - cation | 1.99 |
| exchange resin complex ¹ | |
| Dextroamphetamine Sulfate, USP | 0.089 |
| Amphetamine Aspartate Monohydrate² | 0.128 |
| Mannitol, USP ³ | 65.9 |
| Talc, USP | 1.0 |
| Xanthan gum, NF (Gumixan ™ K) | 0.5 |
| Crospovidone, NF (Kollidon ® CL), | 12 |
| Type A | |
| Microcrystalline cellulose and | 15 |
| Guargum (Avicel ® CE | |
| Sucralose, NF | 1.5 |
| Bubblegum Flavor | 0.5 |
| Silicon dioxide, NF (Syloid ® 244FP) | 0.2 |
| Magnesium stearate, NF | 0.5 |
| Total | 100.0 |

The extended release chewable tablets containing amphetamines are prepared as follows. Weigh the mannitol and microcrystalline cellulose and guar gum (Avicel CE15), and screen through a 20 mesh screen. Add the screened material into ‘V’-Blender. The xanthan gum, Kollidon® CL, sucralose, citric acid, bubble gum flavor, silicon dioxide, talc, Uncoated Amphetamine-cation exchange resin complex, Coated Amphetamine-cation exchange resin complex-matrix, Dextroamphetamine Sulfate and Amphetamine Aspartate Monohydrate are weighed and passed through a #40 mesh screen. The screened material is added into a ‘V’-Blender. Mix the powder blend for 25 minutes. Weigh Magnesium Stearate and pass through #40 mesh screen. Add the screened material into ‘V’-Blender. Mix this powder blend for 5 minutes. The material from the Blender is unloaded and compressed into tablets by adjusting the appropriate tablet weight and hardness.

### Example 13—Efficacy Study of Amphetamine Extended Release (ER) Tablets for Pediatric Use

An amphetamine ER tablet prepared as described in the preceding example, containing the equivalent of 2.5 mg/mL amphetamine base and a ratio of approximately 3:1 of d-amphetamine to l-amphetamine, is tested. The test involves children with attention deficit hyperactivity disorder (ADHD) aged 6-12 years old.

This formulation is assessed to study the attention and behavior of the subjects in a laboratory classroom using the Swanson, Kotkin, Agler, M-Flynn, and Pelham (SKAMP) rating scale. Subjects with ADHD enter a 2-week randomized, double-blind, crossover treatment of the individually optimized dose of the amphetamine ER tablet (2.5 mg/daily to a maximum of 20 mg/daily) or placebo, administered once a day in the morning. At the end of each week, schoolteachers and raters evaluate the attention and behavior of the subjects in a laboratory classroom using the Swanson, Kotkin, Agler, M-Flynn, and Pelham (SKAMP) rating scale. SKAMP scores which are statistically significantly lower (improved) during treatment with the amphetamine ER tablet as compared to placebo are deemed efficacious. The earliest tested time point assessed may be 1 hour post-dose and efficacy may be assessed again at about 13 hours post-dose.

Objective PK measurement occurs over 28 hours post-dose as a primary end point. Pediatric subjects are normally dosed in a manner to provide pharmacologic treatment primarily for the academic time period of the day as well as the after-school period. Two (2) age groups of pediatric subjects may be included in the study: subjects between the ages of 6 to 9 and subjects between the ages of 10 to 12. A total of 12 subjects. This allows the comparison of PK parameters in younger and older pediatric patients treated with a single mid-range dose of amphetamine. A “mid-range” amphetamine dose refers to a comparison of 10 mg amphetamine base to equivalents of other amphetamine products currently FDA approved for the treatment of ADHD in pediatric patients. The study is performed under fasting conditions.

For PK analyses of amphetamine, 9 blood samples are collected in total from each subject. Samples are collected at pre-dose Day 1 (Time 0) and at the following times: Day 1: 1, 3, 4, 6, 8, 10, 12, and 28 hours post dose (Day 2). Time after dosing begins when amphetamine is administered to the subject. With the exception of the 0 (pre-dose) and 28 hour sample, samples are collected within ±5 minutes of the scheduled post-dose time. The pre-dose sample is collected up to 2 hours before dosing. The 28 hour sample is collected 26-30 hours post-dose.

### Example 14—Pharmacological Study with Amphetamine Extended Release (ER) Chewable Tablets Equivalent to 20 mg Amphetamine Free Base

A 20 mg bio-study is conducted using 20 mg AMP ER tablets including (1) chewable tablets under fast and fed conditions, (2) swallowed whole tablets under fed condition, versus suspension under fasted at the same dose (20 mg). The reference drug product is DYANAVEL liquid suspension product [TRIS PHARMA]. An open label, single dose, randomized, four way, cross over relative bioavailability study in 31 healthy subjects is conducted. Results of the pivotal bio study demonstrated bioequivalence between the tablet (independent of mode of administration: chewed or whole) and DYANAVEL AMP suspension reference product in fasted condition. The data also shows increase in the early exposure and also bioequivalence for the chewable tablet in fed condition.

This study compares the bioavailability of the test ER chewable tablet formulation, administered chewed or swallowed intact, to an equivalent 20 mg dose of the reference product (Dyanavel® XR ER Oral Suspension) administered under fasted conditions. Furthermore, the relative bioavailability of the test ER chewable tablet is investigated under fasted and fed conditions in order to assess a possible food effect. This study also assesses the test ER chewable tablet with respect to the dose dumping potential by visual inspection of the individual concentrations versus time profiles. Bioavailability is assessed upon determination of the rate and extent of absorption of a drug in a biological system. AUC and Cmax are common PK parameters obtained to estimate the rate and extent of absorption of a drug product. A sampling schedule was designed for this study to ensure that the AUC, including partial AUCs, and Cmax parameters were adequately assessed from collected plasma. By implementing a crossover design, the estimated PK parameters for each formulation (and respective treatment) are compared within the same subject. Relative bioavailability between the treatment contrasts of interest was determined by a statistical comparison of the AUC, including partial AUCs, and Cmax parameters for d-amphetamine and l-amphetamine.

In each period, subjects received one of the following 4 treatments:
Treatment A—Test Product (as a chewable tablet): one tablet administered after an overnight fast of at least 10 hours
Treatment B—Test Product (as a chewable tablet): one tablet administered 30 minutes after the start of a high-fat, high-calorie breakfast
Treatment C—Test Product (as a whole tablet): one tablet administered after an overnight fast of at least 10 hours
Treatment D—Reference Product: 20 mg (8 mL ER oral suspension [2.5 mg/mL]) administered after an overnight fast of at least 10 hours

Pharmacokinetic analysis is performed on available data from subjects in the PK dataset. The post-dose sample collection times are used in the PK analysis. PK parameters/observations may be estimated for d-amphetamine and l-amphetamine using a non-compartmental approach in Phoenix® WinNonlin® (version 6.4):
AUC0-t: The area under the analyte concentration versus time curve, from time zero (0) to the time of the last measurable analyte concentration (t), as calculated by the linear trapezoidal method.
AUC0-5: The area under the analyte concentration versus time curve, from time zero (0) to 5 hours after dosing, as calculated by the linear trapezoidal method.
AUC0-5 was based on all measurable concentrations within the first 5 hours and is presented as AUC0-5.
AUC5-t: The area under the analyte concentration versus time curve, from 5 hours after dosing to the last measurable analyte concentration (t), as calculated by the linear trapezoidal method.
AUC0-∞: The area under the analyte concentration versus time curve from time zero to infinity. AUC0-∞=AUC0-t+Ct/kel, where Ct is the last measurable analyte concentration.
kel: The apparent first-order elimination rate constant.
Cₘₐₓ: Maximum measured analyte concentration over the sampling period.
Tₘₐₓ: Time of the maximum measured analyte concentration over the sampling period.
AUC0-t/AUC_{0-∞}: The ratio of AUC0-t to AUC0-∞.
TLIN: Start time for linear regression.
LQCT: Last quantifiable concentration time.
R: Correlation coefficient obtained from regression analysis.
Kel, t_{half}, and AUC_{0-∞} parameters were not estimated for concentration-time profiles where the terminal linear phase was not clearly defined.
Individual and mean plasma concentrations versus time curves were plotted.

Descriptive statistics for the PK parameters of d-amphetamine and l-amphetamine are calculated. Descriptive statistics included number of observations, arithmetic mean, SD, geometric mean (where applicable), coefficient of variation (CV), median, minimum, and maximum.

Statistical analysis is performed on quality assured data, with unbalanced groups if necessary, from subjects in the statistical dataset. The PROC GLM procedure from SAS (version 9.4) was used. Analysis of variance (ANOVA) is performed on log-transformed AUC0-5, AUC5-t, AUC0-t, AUC_{0-∞}, and Cmax parameters. The significance of the sequence, period, treatment, and subject(sequence) effects are tested.

Using the same statistical model, the least-squares-means, the differences between the treatments least-squares-means and the corresponding standard errors of these differences are estimated for log-transformed AUC0-5, AUC5-t, AUC0-t, AUC0-∞, and Cmax parameters.

Based on these statistics, the ratios of the geometric means for treatments and the corresponding 90% CIs are calculated for the following comparisons:

### Primary Objectives:

Relative Bioavailability (Treatment A versus Treatment D; Treatment C versus Treatment D); and
Food Effect (Treatment B versus Treatment A).

### Secondary Objectives:

Relative Bioavailability (Treatment A versus Treatment C).

See, FIGS. 1A-1D and FIGS. 2A-2D.

### Example 15: Effect of a Single Dose of Amphetamine Extended-Release Tablet on Driving Performance in Young Adult Drivers with ADHD: A Randomized, Double-Blind, Placebo-Controlled Study Using a Laboratory Driving Environment

Laboratory driving environments provide the best alternative for addressing questions about the etiology of impaired driving behaviors in at-risk populations that are too dangerous or expensive to evaluate in on-road tests. Adler, L. A., Resnick, S., Kunz, M., & Devinsky, O. (1995). Open-label trial of venlafaxine in adults with attention deficit disorder. Psychopharmacology Bulletin, 31(4), 785-788. Simulators provide reproducibility, control, efficiency, and ease of use that are lacking in on-road behavior evaluations. Godley, S. T., Triggs, T. J., & Fildes, B. N. (2002). Driving simulator validation for speed research. Accid Anal Prev, 34(5), 589-600. Since driving simulation is a highly specialized field, the development of a valid driving simulation for ADHD requires the collaboration of clinical experts in ADHD and engineers in the field of transportation safety and driving simulation for paradigm validation. Moreover, for driving simulators to be valid instruments for collecting data on driving behavior, correspondence between virtual and real driving must be established. This can be achieved by demonstrating that the simulator duplicates differences between ADHD and non-ADHD groups that have been recorded by other methods (e.g., driving histories) known to be valid measures of the construct of interest (i.e., driving performance). Reimer, B., D'Ambrosio, L. A., Coughlin, J. E., Kafrissen, M. E., & Biederman, J. (2006). Using self-reported data to assess the validity of driving simulation data. Behav Res Methods, 38(2), 314-324.

Objective: ADHD is a risk factor for motor vehicle accidents, including injuries and fatalities, traffic citations and license suspension. This study assessed the impact of a single dose of amphetamine extended-release (DYANAVEL® XR) tablets (AMPH ER TAB) on driving performance in young adult drivers with ADHD in a laboratory-based simulation environment.

Methods: This single-center, randomized, double-blind, placebo-controlled, parallel study evaluated driving performance at predose, 45-minutes and 10-hours postdose, emulating typical morning and evening commutes, and based on AMPH ER efficacy, with efficacy observed as early as 30 minutes and as late as 13 hours postdose. The study evaluated relative crash risk of drivers with ADHD who took a single dose AMPH ER TAB (20 mg). The primary endpoints were the difference from placebo of the Minimum Time to Collision, Braking Percent, and Highest Braking Intensity. The key secondary endpoint was Total Crash Count during the last drive.

Results: Forty-one male and female drivers aged 18-25 years who met DSM-V criteria for ADHD completed the study. Examination of driving performance during postdose drives demonstrated that Median Minimum Time to Collision and Maximum Highest Braking Intensity were statistically significantly improved with AMPH ER TAB at both 45-minutes and 10-hours postdose. Generally, drivers with ADHD who received AMPH ER TAB had numerically improved outcomes across all measures. Single dose of AMPH ER TAB was well tolerated with no dropouts due to adverse events.

Conclusions: Drivers with ADHD who took AMPH ER TAB showed improved behavior in the driving simulation associated with a lower crash risk relative to subjects who took placebo.

### INTRODUCTION

Driving involves many cognitive abilities and executive functions (e.g., estimating time, alertness, focus). The inattention and impulsivity inherent to ADHD can interfere with driving and contribute to an increased likelihood of traffic citations, vehicular crashes and license suspensions. American Psychiatric Association. Diagnostic and statistical manual of mental disorders (DSM-5). APA Publications, 22 May 2013; Danielson M L, Bitsko R H, Ghandour R M, et al. Prevalence of parent-reported ADHD diagnosis and associated treatment among U.S. children and adolescents. J Clin Child Adolesc Psychiatry. 2018; 47(2):199-212.

### Methods: Driving Simulations:

This unique study was conducted using a driving simulator developed and validated to assess aspects of ADHD that could account for driving impairments. [Biederman, J., Fried, R., Monuteaux, M. C., Reimer, B., Coughlin, J. F., Surman, C. B., Aleardi, M., Dougherty, M., Schoenfeld, S., Spencer, T. J., & Faraone, S. V. (2007). A laboratory driving simulation for assessment of driving behavior in adults with ADHD: a controlled study. Annals of General Psychiatry, 6, 4. https:_doi_org/10_1186/1744-859X-6-4. The simulation—done in a real vehicle with a virtual roadway projected on a large, single screen—tests driving under various environments (urban, rural, highway), under differing stimulus intensity (active and monotonous), and while performing other tasks (e.g., driving while having a cellphone conversation).
Each driver participated in 3 simulated drives. The first drive was prior to dosing and served as a baseline and familiarization to the simulator in order to reduce the novelty/gamification of the experience for the subsequent drives. The second and third drives took place 45 minutes and 10 hours post-dose.
The simulation vehicle was a Volkswagen Beetle, with 3 large screens that wrapped around the front, a screen at the rear and screens placed in the side view mirrors.
The vehicle was placed on a moving base that provided realistic tilting when braking and steering.

TABLE 1
Primary and Key Secondary Endpoints
Primary Endpoints

| | |
|---|---|
| Time to | How close the participant's vehicle came |
| Collision | to colliding with a lead vehicle |
| (in seconds) | |
| Brake Percent | The percentage of the events in which |
| | the brake was applied |
| Brake | Range is 0 (no depression) to 300 (full |
| Intensity | depression of the brake pedal) |

Secondary Endpoints

| | |
|---|---|
| Crash Count | The number of crashes observed in each |
| | group (only present at the end of Drive 3) |

Descriptive analyses were conducted to evaluate the mean, median and standard error.

Each drive took approximately 26 minutes during which drivers experienced 8 active response events, and 6 distractor events. The third drive included a special event at the end that required hard braking to avoid a crash.
Active Response Events required drivers to slow their vehicle. There were 4 event types (2 of each per drive) with the order of events varying during each drive.
Distractor events were similar to active events but did not evolve into a situation where an active response was required of the driver

### Risk of Being Rear-Ended by a Following Vehicle (Primary Endpoints)

If a driver brakes suddenly to avoid crashing into a vehicle in front of them, there is a risk that a vehicle traveling behind them may not stop in time and thus rear-end the driver. Drivers generally brake hard either because they failed to notice an emerging safety critical event in front of them in sufficient time to manage the situation by a less drastic response (reducing speed by backing-off the accelerator or more gradual braking) or if they were not paying attention and were startled by the event, potentially braking harder than was necessary. All of the scenarios presented during the driving simulation could be managed by reasonably attentive drivers either without use of the brake (anticipatory slowing by backing-off on the accelerator/throttle) or with low force application of the brake early in the scenario.

### Variable List: Presence of Braking

Braking Percent—The number of events in which the participant responded to the event by pressing the brake, expressed as the percentage of the events in which the brake was applied.

### Highest Braking Intensity

This variable considered the maximum amount of momentary brake force applied during an event. The units for brake force were on a relative scale ranging from 0 (no depression of the brake pedal) to 300 (full depression of the brake pedal).
Highest Braking Intensity—Largest momentary value across all events
Highest Braking Intensity (Mean)—A composite variable consisting of the mean of the largest momentary value from each event.

Interpretive Summary: Larger highest braking intensity values indicate behavior that is associated with a greater risk of being rear-ended; conversely, all other things being equal, the smaller the value, the greater the margin of safety that is present in the participant's driving behavior—smaller values are generally desirable.

The braking percent variable is a more simplistic, binary consideration of whether the participant used the brake or not during each event. Again, smaller values can be seen as generally desirable as long as a participant did not come close to crashing into the lead vehicle.

(Overall, use of the brake was found to be quite frequent with participants using the brake in approximately 82% of the events during the first drive.)

### Overt Reaction Times (Other Active Response Endpoints)

These measures attempt to assess how quickly participants anticipated the potential for a conflict in the driving environment and engaged in an active response to reduce the likelihood of a crash. As noted above, all of the scenarios could be managed by reasonably attentive drivers either without use of the brake (anticipatory slowing by backing-off on the accelerator/throttle) or with low force application of the brake early in the scenario. The unit of measurement is in seconds.

### Variable List: Reaction Time:

Throttle Release Time—This is a measure of how many seconds into the event the participant began slowing by taking their foot off the accelerator/throttle. This is a composite variable consisting of the mean across all events of the point in time at which this occurred, if it did. Smaller values are desirable as they indicate greater reactive attentiveness.
Brake Reaction Time—This is a measure of how many seconds into the event the participant began slowing by pressing the brake. This is a composite variable consisting of the mean across all events of the point in time at which this occurred. Smaller values are desirable as they indicate greater reactive attentiveness.
Note: A value of 0 was entered as the brake reaction time for events when participants did not brake. However, some caution must be exercised in interpreting
0 values. Ideally, not actively braking could be associated with the participant effectively managing the event by releasing the throttle early in the event such that braking was not required. Alternatively, it could be associated with a failure to actively respond to the situation.
   Interpretive Summary: For each of the above measures, smaller values indicate faster active responses to the event and larger values indicate slower active responses to the event. All other things being equal, smaller/faster response values are suggestive of an earlier awareness of a potentially dangerous event unfolding (i.e., mind wandering) and an earlier engagement in a proactive response to minimize the likelihood of conflict.
Mean Speed—The mean of the velocity of the participant's vehicle during the event window in miles per hour. Higher speeds are potentially riskier, but no a priori expectation regarding potential medication effects was hypothesized for this study.
Standard Deviation of Speed—The mean of the standard deviation of the speed (velocity) of the participant's vehicle during the active event window in miles per hour. Higher standard deviation values are generally interpreted as reflecting less attentive control of the vehicle, but no a priori expectation regarding potential medication effects was hypothesized for this study.
Standard Deviation of Lane Position—The mean of the standard deviation of the velocity of the vehicle's lane position during the active event window in meters. Higher standard deviation of lane position values are generally interpreted as reflecting less attentive control of the vehicle, but no a priori expectation regarding potential medication effects was hypothesized for this study.
Steering Wheel Reversals—Both “small” and “large” steering wheel reversals were counted during each event. Higher steering reversal values are generally interpreted as reflecting less attentive control of the vehicle, but no a priori expectation regarding potential medication effects was hypothesized for this study. “Large” steering wheel reversals turned out to be very rare in the dataset and were not amenable to statistical analysis. Consequently, the variable “steering wheel reversals” considers only values within the “small steering wheel reversal” range. (See Appendix A for a technical description of how these values are calculated.)
Lane Excursion Count—A simple count of the number of times the participant's vehicle crossed out of lane (see Appendix A for technical details). Higher values may be associated with a need to swerve out of lane due to not having time to respond to an emerging event by reducing speed through throttle and/or brake control. This is generally associated with either a failure to notice an emerging event until late in the period or what would generally be considered a more reckless style of driving.
   Interpretive Summary: For each of the above measures, higher values are generally associated with riskier behavior and/or less attentive control of the vehicle. Lower values are generally considered as more desirable, but no a priori expectation regarding potential medication effects were hypothesized for these variables for this study.

| Variable | Ideal Value | | |
|---|---|---|---|
| Minimum Time to Collision | Larger | ↑ | |
| Minimum Time to Collision (Mean) | Larger | ↑ | |
| Minimum Time to Collision (Median) | Larger | ↑ | |
| Braking Percent | Smaller | | ↓ |
| Highest Braking Intensity | Smaller | | ↓ |
| Highest Braking Intensity (Mean) | Smaller | | ↓ |
| Throttle Release Time | Sooner | | ↓ |
| Brake Reaction Time | Sooner | | ↓ |
| Mean Speed | Smaller | | ↓ |
| Standard Deviation of Speed | * | | * |
| Standard Deviation of Lane Position | Smaller | | ↓ |
| Steering Wheel Reversals | Smaller | | ↓ |
| Lane Excursion Count | Smaller | | ↓ |

Higher values for the minimum time to collision measures represent less risky behavior (greater separation from a lead vehicle in terms of time to collision). For all of the remaining variables, lower values are generally interpreted as representing less risky driving behavior. *As noted above, while smaller standard deviation of speed values are generally interpreted as representing less risky behavior, due to the range of speed adjustments that might be relatively safe as opposed to failing to adjust speed in dealing with emerging events, no specific a priori expectation was hypothesized for this study and this variable was collected for exploratory purposes.

Driver Demographics

| | | Safety and | |
|---|---|---|---|
| | | Randomized | |
| Demographic | | Population | |
| Characteristic | Results | (N = 41) | |
| Sex | Female | 20 | (48.8%) |
| | Male | 21 | (51.2%) |
| Age (years) | Mean ± SD | 22 ± 2 | |
| | Median | 22 | |
| | Range | 18-25 | |
| Weight (kg) | Mean ± SD | 72.21 ± 16.48 | |
| Height (cm) | Mean ± SD | 171.10 ± 9.50 | |
| Race, n (%) | Asian | 2 | (4.9%) |
| | Black or African American | 3 | (7.3%) |
| | Other - Mexican | 1 | (2.4%) |
| | Other - White/Asian | 1 | (2.4%) |
| | White | 34 | (82.9%) |
| Ethnicity, | Hispanic/Latino | 7 | (17.1%) |
| n (%) | Non-Hispanic/Latino | 34 | (82.9%) |

Key Inclusion Criteria:
Licensed Drivers
Aged 18 to 25 years
Diagnosis of ADHD based on DSM-V
Key Exclusion Criteria:
Current active symptoms of other psychiatric disorders (Drivers were not excluded if they were taking concomitant SSRIs or SNRIs)
History of cardiovascular comorbidities
History or presence of uncontrolled hypertension at screening
Use of ADHD medications including all stimulants within 48 hours of the Driving Simulator Visit

### Results:

### Statistical Methods

Since the assumptions required for applying the MMRM model could not be fulfilled, alternative statistical models were be applied. More particularly, the statistical assumption of normality was assessed for each continuous observation of each outcome using Shapiro-Wilk tests. If the assumption was violated for one or more of the distributions across the observations, then non-parametric Friedman's ANOVA tests were used to test for significant changes across time. When a significant main effect was detected, Dunn's tests were used to analyze post hoc differences amongst the observations. Medians and interquartile ranges were reported and interpreted for the non-parametric comparisons. When normality was met for each observation of an outcome, repeated-measures ANOVA was used to test for significant change across time. Means and standard deviations were reported for any parametric ANOVA tests. All analyses were performed using SPSS Version 29 (Armonk, NY: IBM Corp.) and statistical significance was assumed at a Bonferroni-adjusted value of 0.00625 for statistical significance.

### Statistical Results

There were violations of normality for 12 out of 13 outcomes (only Mean SD Velocity met the assumption for all three observations), so non-parametric Friedman's ANOVA was used to test for statistical significance for the majority of the outcomes. For the primary outcomes, there were no significant main effects detected for TOC Mean Min, X²(2)=5.06, p=0.08, TOC Median Min, X²(2)=3.29, p=0.19, Break Percent, X²(2)=1.24, p=0.54, Mean Brake RT Zeros, X²(2)=1.16, p=0.56, or Mean Velocity, X²(2)=5.77, p=0.06. There was a significant main effect detected for TOC Min, X²(2)=12.12, p=0.002. Post hoc tests showed a significant increase from baseline to 10 hours, p=0.002, but not for baseline to 45 minutes, p=0.08. There was a significant main effect for Mean Max Brake Force, X²(2)=11.38, p=0.003, and a significant post hoc effect was found between baseline and 10 hours, p=0.002, but not for baseline to 45 minutes, p=0.47. A significant main effect was detected for Mean Max Brake Zeros, X²(2)=10.71, p=0.005, and the post hoc significance was found between baseline and 10 hours, p=0.003, but not between baseline and 45 minutes, p=0.18.

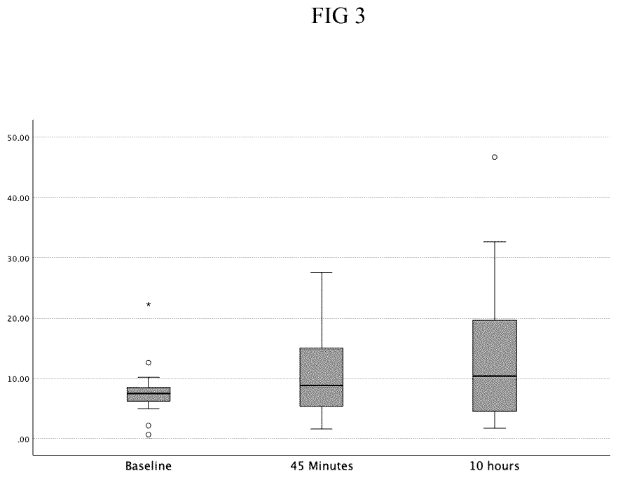

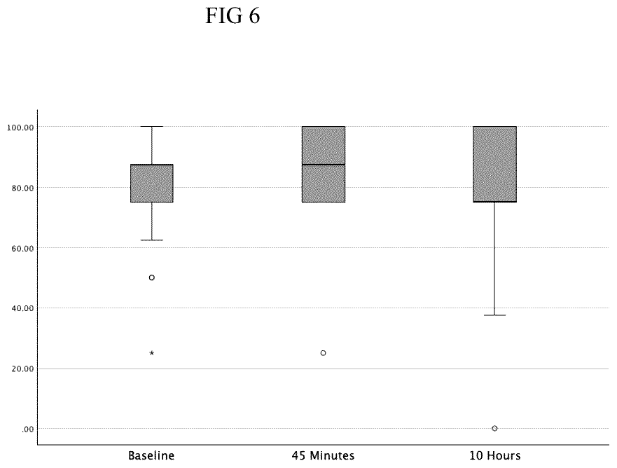

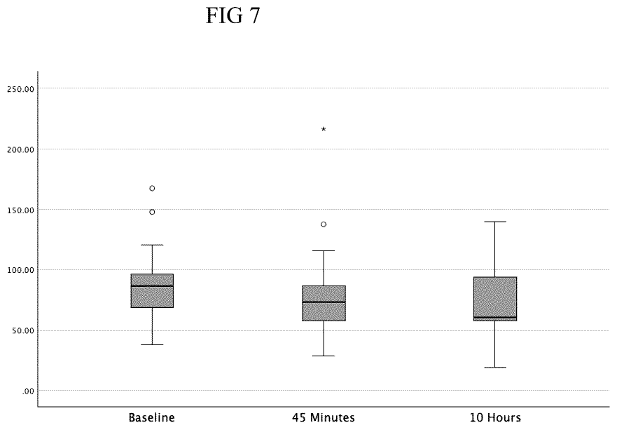

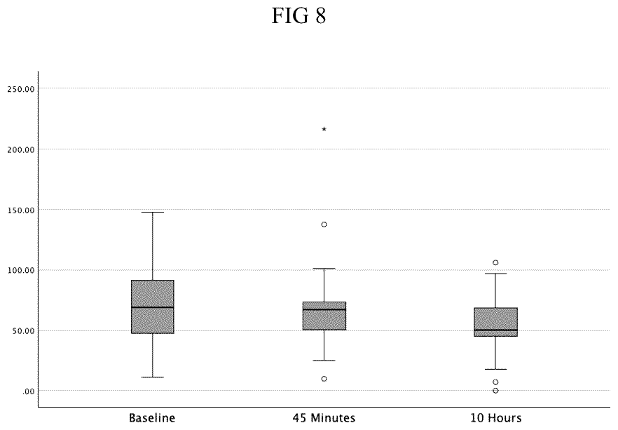

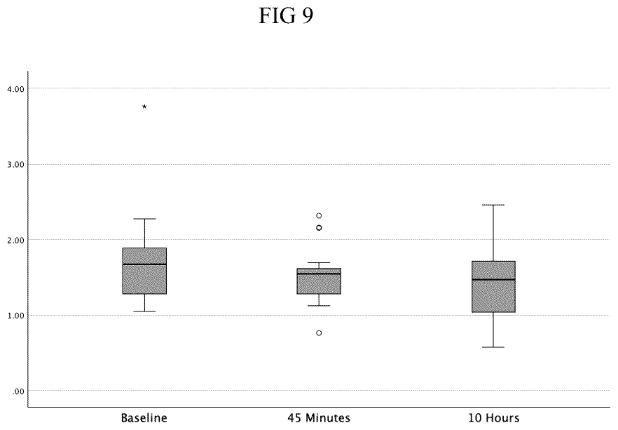

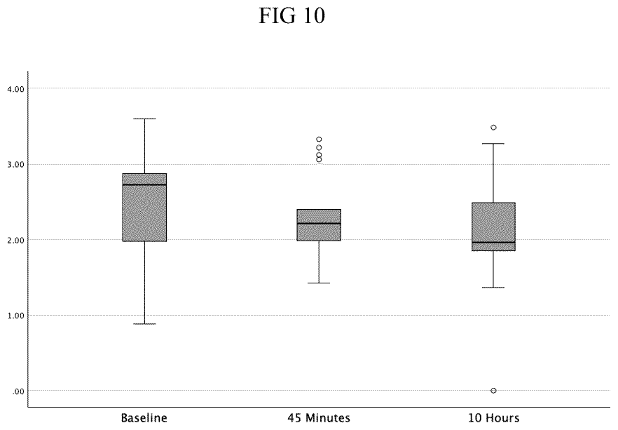

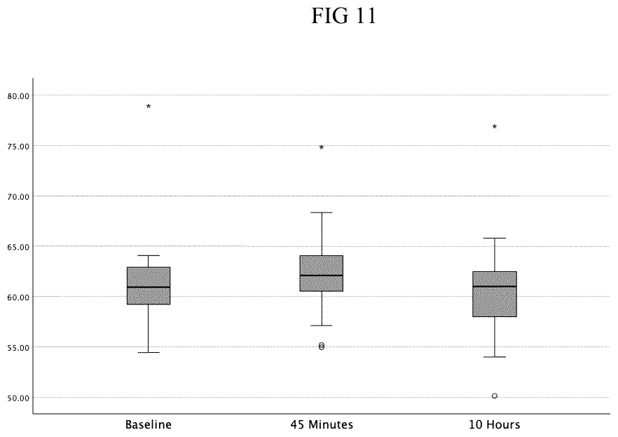

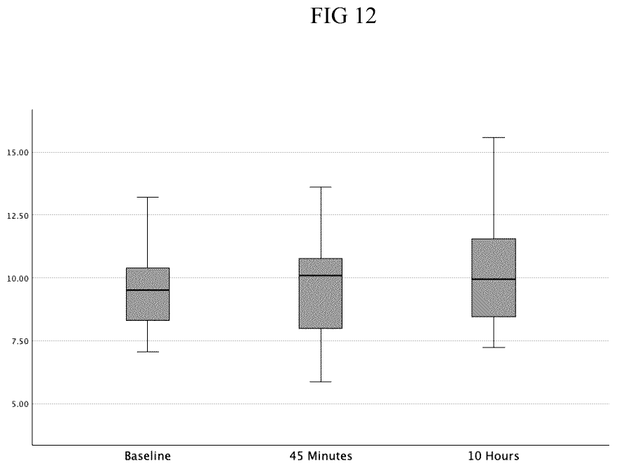

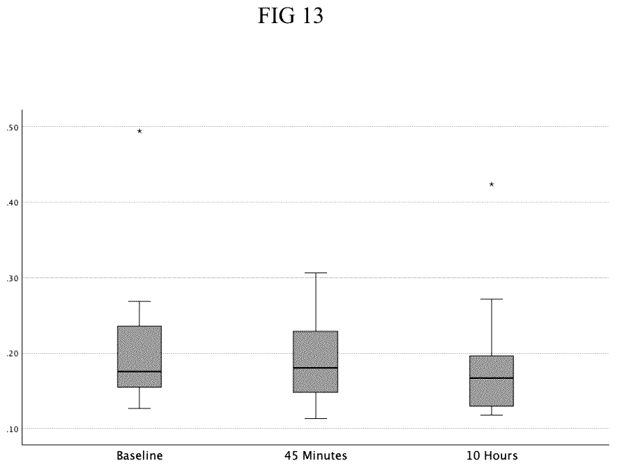

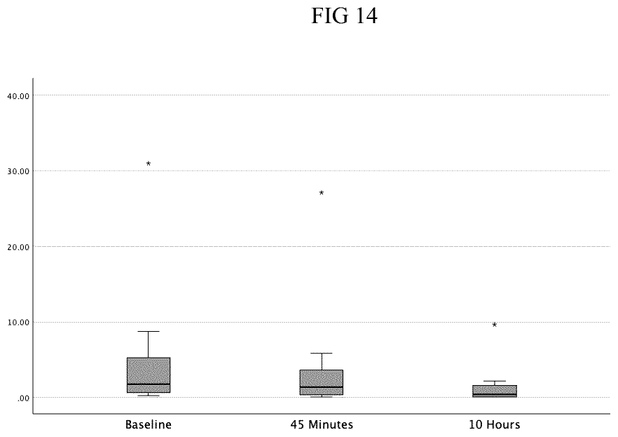

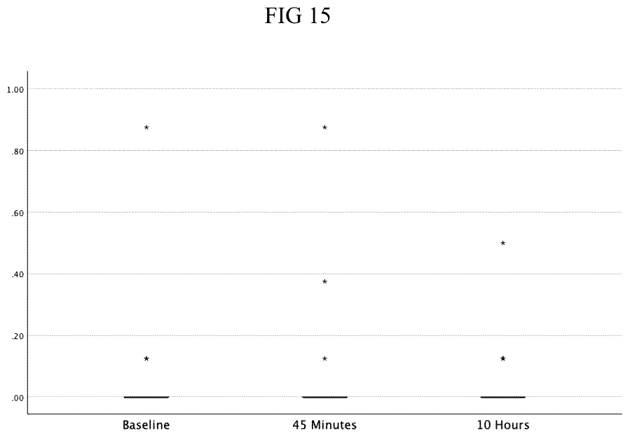

In regard to the secondary outcomes, no significant main effects were detected for Mean Throttle Release RT, X²(2)=2.94, p=0.23, Mean SD Velocity, F(2,15)=3.29, p=0.07, or Mean Lane Excursion Count, X²(2)=0.00, p=1.00. The main effect for Mean SDLP was statistically significant, X²(2)=11.41, p=0.003, and there was a significant post hoc difference was observed between baseline and 10 hours, p=0.003, but no difference was found between baseline and 45 minutes, p=0.049. Finally, there was a statistically significant main effect found across time for Mean Small WR, X²(2)=20.86, p<0.001, and the post hoc significance was found between baseline and 10 hours, p<0.001, but not between baseline and 45 minutes, p=0.03. See Table below for all of the descriptive statistics associated with the within-subjects comparisons. FIGS. 3-16 present boxplots for each comparison.

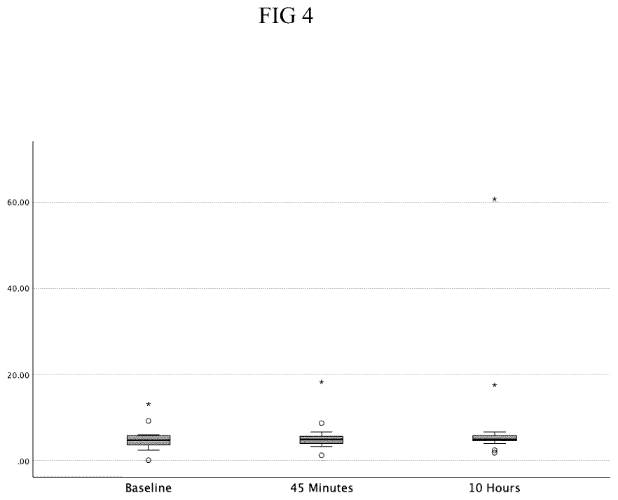

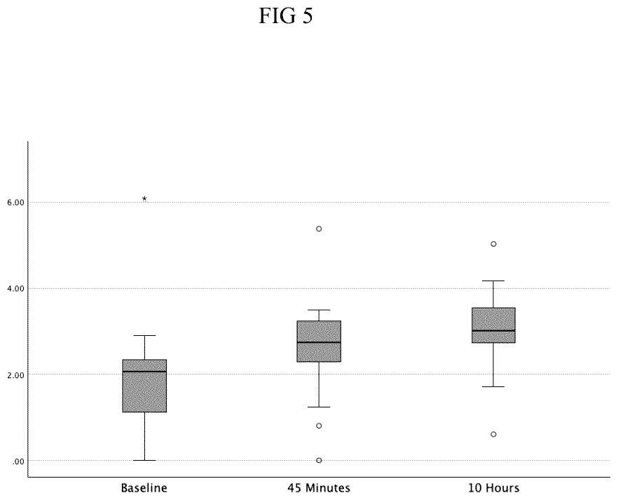

TABLE
Within-subjects Comparisons of Driving Variables

| Outcome | Baseline | 45 minutes | 10 hours | p-value |
|---|---|---|---|---|
| TOC Mean Min* | 7.42 | 8.86 | 10.32 | 0.08 |
| (n = 17) (See FIG. 3) | (5.63-8.77) | (5.18-15.38) | (4.84-20.17) | |
| TOC Median Min* | 4.75 | 4.97 | 4.91 | 0.19 |
| (n = 17) (See FIG. 4) | (3.65-5.89( | (3.79-5.95) | (4.44-6.09) | |
| TOC Min* | 2.07 | 2.74 | 3.00 | 0.002*** |
| (n = 17) (See FIG. 5) | (1.11-2.49) | (2.02-3.25) | (2.33-3.57) | |
| Brake Percent* | 87.50 | 87.50 | 75.00 | 0.54 |
| (n = 17) | (68.75-93.75) | (75.00-100.00) | (68.75-100.00) | |
| Mean Max Brake | 86.17 | 73.20 | 60.33 | 0.003** |
| Force* (n = 16) | (67.99-96.56) | (57.32-88.15) | (57.47-95.09) | |
| Mean Max Brake | 69.02 | 67.16 | 49.92 | 0.005*** |
| Force Zeros* (n = 17) | (46.10-93.94) | (42.77-73.60) | (44.43-69.17) | |
| Mean Throttle Release | 1.67 | 1.54 | 1.47 | 0.23 |
| RT* (n = 17) | (1.26-1.98) | (1.25-1.66) | (1.03-1.72) | |
| Mean Brake RT | 2.72 | 2.21 | 1.96 | 0.56 |
| Zeros* (n = 17) | (1.97-2.88) | (1.87-2.73) | (1.75-2.56) | |
| Mean Velocity* | 60.92 | 62.11 | 61.01 | 0.06 |
| (n = 17) | (58.90-63.05) | (58.95-65.25) | (57.74-62.57) | |
| Mean SD Velocity** | 9.46 (1.76) | 9.53 (1.95) | 10.22 (2.28) | 0.07 |
| (n = 17) | | | | |
| Mean SDLP* (n = 17) | 0.18 | 0.18 | 0.17 | 0.003*** |
| | (0.15-0.24) | (0.14-0.24) | (0.13-0.20) | |
| Mean Small SWR* | 1.75 | 1.38 | 0.38 | <0.001*** |
| (n = 17) | (0.56-5.69) | (0.31-4.00) | (0.00-1.81) | |
| Mean Lane Excursion | 0.00 | 0.00 | 0.00 | 1.00 |
| Count* (n = 17) | (0.00-0.00) | (0.00-0.00) | (0.00-0.07) | |

Note:
*Values are median (interquartile range),
**values are mean (standard deviation),
***p < 0.05, statistically significant main effect; TOC: Time to Collision; RT: Reaction Time; SD: Standard Deviation; SDLP: Standard Deviation of Lane Position; SWR: Steering Wheel Reversals.

### Conclusions:

Patients with clinically established ADHD who took AMPH ER TAB showed a pattern of behavior in the driving simulation associated with a lower relative crash risk than participants who took the placebo.
Driving studies serve an important role in understanding the daily impact of ADHD on patients and the public at large.
This study employed a methodology that reflected driving in the real world and the impact AMPH ER TAB had on morning and afternoon drives suggesting a safer commute for both drivers with ADHD and other drivers on the road.
As an exploratory study, the number of patients enrolled, the limited dosing regimen, and the lack of normalized driving data warrant further research to better understand the outcomes measured and the clinical application of such studies.
¹Barkley et al. J Safety Res 2007. 38(1) 113-28.
²Amanda, et al. J Pediatric Psychol 2007
³Childress A C et al. J Child Adolesc Psychopharmacol. 2018. 28(5) 306-313.
⁴Childress A C et al. J Child Adolesc Psychopharmacol. 2019. 29(1) 1-7.
⁵Cutler A J, et al. J Clin Psychiatry. 2022. 83(5):22,14438.
⁶Kando J et al. Poster presented at AMCP 2020, Virtual, 21-24 Apr. 2020.

All patents, patent publications, and other publications listed in this specification, as well as U.S. Patent Application No. 63/487,158, filed Feb. 27, 2023, are hereby incorporated by reference in their entirety. While the invention has been described with reference to a particularly preferred embodiment, it will be appreciated that modifications can be made without departing from the spirit of the invention. Such modifications are intended to fall within the scope of the appended claims.

1. A method for improving driving behavior in a human subject having attention deficit hyperactivity disorder, said method comprising dosing the subject once daily with an amphetamine extended release tablet,
wherein the tablet provides a single plasma concentration peak for d-amphetamine and for l-amphetamine, wherein the tablet lacks a pH-dependent coating which provides delayed release to an amphetamine component, and wherein the tablet further comprises:
(A) a modified release amphetamine component which comprises at least one modified release barrier coated amphetamine-cation exchange resin complex-optional matrix which comprises (i) two or more amphetamines bound to the same cation exchange resin, wherein when the optional matrix is present, the amphetamine-cation exchange resin complex-matrix further comprises a hydrophilic polymer or copolymer or a hydrophobic polymer and (ii) a pH-independent barrier coating which provides a modified release to the two or more amphetamines, wherein the two or more amphetamines are at least a d-amphetamine and an l-amphetamine, wherein the d-amphetamine and the l-amphetamine are provided by d-amphetamine and at least one of (d,l)-amphetamine and l-amphetamine, wherein the modified release amphetamine component (A) comprises at least 5% w/w of the total amphetamines in the tablet based on the weight of free amphetamine base; and
(B) immediate release amphetamine components which comprise greater than 70% w/w of the total amphetamines in the tablet based on the total weight of free amphetamine base in the tablet, and wherein the immediate release amphetamine components further comprise:
(i) a first immediate release amphetamine component which comprises d-amphetamine or a pharmaceutically acceptable salt thereof, and l-amphetamine or a pharmaceutically acceptable salt thereof, or mixtures thereof, wherein the d- and l-amphetamine are provided by d-amphetamine and at least one of (d,l)-amphetamine and l-amphetamine, wherein the immediate release amphetamine component B(i) comprises at least 5% w/w of the total amphetamines in the tablet based on the weight of free amphetamine base;
(ii) amphetamine aspartate; and
(iii) dextroamphetamine sulfate.

2. The method according to claim 1, wherein the patient is provided with a single daily dose of the tablet.

3. The method according to claim 1, wherein the tablet comprises 20 mg amphetamines.

4. The method according to claim 1, wherein the driving behavior of the patient is improved at both 45-minutes and 10-hours post-dose of the tablet.

5. The method according to claim 1, wherein the driving behavior of the patient is improved as early as 30 minutes and as late as 13-hours post-dose of the tablet.

6. The method according to claim 1, wherein improved driving behavior is characterized by one or more of: lower crash risk, increased maximum highest braking intensity, maximum shortened braking time, increased median minimum time to collision, improved lane position and/or decreased lane deviation

7. The method according to claim 1, wherein the amphetamines in first and second immediate release amphetamine component of (B) are about 80% w/w of the amphetamines in the total tablet.

8. The method according to claim 1, wherein the amphetamines in the first immediate release amphetamine component of (B)(i) are about 20% w/w of the total amphetamines in the tablet.

9. The method according to claim 1, wherein the amphetamines in the second immediate release amphetamine component (B)(ii) are about 60% w/w of the total amphetamines in the tablet.

10. The method according to claim 1, wherein the modified release component (A) further comprises a water insoluble polymer or copolymer or a hydrophilic polymer which forms a matrix with the amphetamine-cation exchange resin complex of (i).

14. The method according to claim 1, wherein the modified release component (A) comprises (d,l)-amphetamine and d-amphetamine.

15. The method according to claim 1, wherein the immediate release component (C) comprises (d,l)-amphetamine and d-amphetamine.

16. The method according to claim 1, wherein the immediate release component (B) comprises: (a) (d,l)-amphetamine or a pharmaceutically acceptable salt thereof, and d-amphetamine or pharmaceutically acceptable salt thereof, or a mixture thereof; or (l)-amphetamine or a pharmaceutically acceptable salt thereof, (d)-amphetamine or a pharmaceutically acceptable salt thereof, or mixtures thereof, optionally further comprising (d,l)-amphetamine or a salt thereof.

17. The method according to claim 1, wherein the tablet comprises at least two different amphetamine salts counterions.

19. The method according to claim 1, wherein the tablet is a chewable tablet.

20. The method according to claim 1, wherein the barrier coating is a cured, water-insoluble, water-permeable, non-ionic, pH-independent barrier coating comprises about 70 to about 90% w/w polyvinylacetate, a stabilizer, and about 2 to about 10% w/w of a plasticizer.

21. The method according to claim 1, wherein said tablet does not exceed 500 mg in total weight.

22. The method according to claim 1, wherein each of the (d,l)-amphetamine and the d-amphetamine of modified release component (A) is bound to a separate cation exchange resin and/or each of the (d,l)-amphetamine and the d-amphetamine of the immediate release component (C) is bound to a separate cation exchange resin.

23. A method of decreasing the risk of a driving accident in an adult human having attention deficit hyperactivity disorder and operating a motor vehicle, said method comprising providing the adult with an amphetamine extended release tablet,
wherein a single plasma concentration peak for d-amphetamine and for I-amphetamine, wherein the tablet further comprises:
(A) a modified release amphetamine component which comprises at least one modified release barrier coated amphetamine-cation exchange resin complex-optional matrix which comprises (i) (d,l)-amphetamine and l-amphetamine bound to the same cation exchange resin or each bound to a different cation exchange resin, wherein when the optional matrix is present, the amphetamine-cation exchange resin complex-matrix further comprises a hydrophilic polymer or copolymer or a hydrophobic polymer and (ii) a water-insoluble, water-permeable, pH-independent, barrier coating which provides a modified release to the amphetamines, wherein the barrier coating comprises polyvinyl acetate and a plasticizer; and
wherein the ratio of d-amphetamine to l-amphetamine is about 3.2 to about 1;
(B) immediate release amphetamine components which comprise greater than 60% w/w of the total amphetamines based on the total weight of free amphetamine base in the tablet, and wherein the immediate release amphetamine components are (i), (ii) and (iii):
(i) an immediate release amphetamine-cation exchange resin complex in an optional matrix, wherein the amphetamine-cation exchange resin complex-optional matrix comprises (d,l)-amphetamine and l-amphetamine both bound to the same cation exchange resin; and
(ii) an amphetamine aspartate; and
(iii) a dextroamphetamine sulfate.

24. The method of claim 23, wherein the amphetamine tablet provides improved driving from about 45 minutes post-dosing to about 10 hours post dosing.

25. The method of claim 23, wherein the tablet is scored and chewable.

26. The method of claim 23, wherein the tablet comprises a dose of 5 mg amphetamine, as calculated based on the amount of free amphetamine base.

27. The method of claim 23, wherein the tablet comprises a dose of 20 mg amphetamine, as calculated based on the amount of free amphetamine base.

28. The method according to claim 23, wherein the motor vehicle is an automobile.

29. The method according to claim 23, wherein the subject is 16 to 60 years old.

30. The method according to claim 23, wherein the subject is 18 to 25 years old.
